(19) United States
(12) Patent Application Publication
(10) Pub. No.: US 2025/0283022 A1
(43) Pub. Date: Sep. 11, 2025

(54) SYSTEMS AND METHODS FOR RETRIEVING CELLS FROM A CONTINUOUS CULTURE MICROFLUIDIC DEVICE

(71) Applicant: President and Fellows of Harvard College, Cambridge, MA (US)

(72) Inventors: Johan Paulsson, Cambridge, MA (US); Scott Luro, Cambridge, MA (US)

(73) Assignee: President and Fellows of Harvard College, Cambridge, MA (US)

(21) Appl. No.: 18/860,755

(22) PCT Filed: Apr. 27, 2023
(86) PCT No.: PCT/US2023/020167

§ 371 Date: Oct. 28, 2024

Related U.S. Application Data
(60) Provisional application No. 63/336,997, filed on Apr. 29, 2022.

(51) Int. Cl. C12M 3/06; C12M 1/00; C12M 1/34

(52) U.S. Cl. CPC C12M 23/16; C12M 29/00; C12M 41/06; C12M 41/10; C12M 41/44; C12M 47/04; C12M 47/16

(57) ABSTRACT

The present disclosure is generally directed to systems and methods for retrieving cells from a continuous culture microfluidic device. In some aspects, a system that allows for selective extraction of one or more cells of interest from an arbitrary population of cells using a high-throughput negative cell selection technique is disclosed herein. For example, the system may comprise a microfluidic device comprising a plurality of cell growth trenches configured to contain cells and a patterned light source capable of selectively killing unwanted cells contained within the device. Coupled with time-lapse imaging, one or more cells of interest within the device may, in some aspects, be identified and extracted with a relatively high extraction efficiency, e.g., at least 99.9% of cells of interest may be extracted from the plurality of cells. In addition, some aspects of the disclosure are directed to methods for using such a system.

### RELATED APPLICATIONS

This application claims the benefit of U.S. Provisional Patent Application Ser. No. 63/336,997, filed Apr. 29, 2022, entitled “Systems and Methods for Retrieving Cells from a Continuous Culture Microfluidic Device,” incorporated herein by reference in its entirety.

### FIELD

Certain aspects of the present disclosure are generally directed to systems and methods for retrieving cells from a continuous culture microfluidic device.

### BACKGROUND

Cell screenings play a fundamental role in biology and makes it possible to identify one or more cells of interest based on a desired phenotype and/or genotype associated with the target cells. Current cell screening techniques only provide endpoint low-resolution snapshots, and offer little information about growth, intracellular dynamics, and responses to environmental changes. Furthermore, because each cell is probed only once, current techniques struggle to distinguish genetically stable properties from transient phenotypic heterogeneity. In some cases, current techniques are often limited by scalability, screening errors due to phenotypic mischaracterization, etc. Thus, more effective system and methods for cell screening and retrieval are still needed.

### SUMMARY

Certain aspects of the present disclosure are generally directed to systems and methods for retrieving cells from a continuous culture microfluidic device. The subject matter of the present disclosure involves, in some cases, interrelated products, alternative solutions to a particular problem, and/or a plurality of different uses of one or more systems and/or articles.

One aspect of the present disclosure is generally direct to a system. According to one set of embodiments, the system comprises a microfluidic device comprising a cell flow layer comprising a growth channel, a plurality of cell growth trenches fluidically coupled to the growth channel, and a control layer configured to control flow of a fluid in the cell flow layer; a laser positioned to direct light at at least a portion of the cell growth trenches; and an electronically reconfigurable mask positioned to selectively shield the light from the laser. In some embodiments, the growth channel comprises an inlet portion, an outlet portion, an inlet valve portion associated with the inlet portion, and an outlet valve portion associated with the outlet portion.

According to another set of embodiments, the system comprises a microfluidic device comprising a cell flow layer comprising a growth channel, a plurality of cell growth trenches fluidically coupled to the growth channel, and a control layer configured to control flow of a fluid in the cell flow layer; and a laser positioned to direct light at at least a portion of the cell growth trenches, wherein the laser is configured to produce light having an intensity capable of killing one or more cells, and a wavelength less than or equal to 1000 nm. In some embodiments, the growth channel comprises an inlet portion, an outlet portion, an inlet valve portion associated with the inlet portion, and an outlet valve portion associated with the outlet portion.

According to yet another set of embodiments, the system comprises a microfluidic device comprising a plurality of single-entry, single-file cell growth trenches fluidically coupled to a growth channel; a laser positioned to direct light at at least a portion of the cell growth trenches; and an electronically reconfigurable mask positioned to selectively shield the light from the laser.

According to yet another set of embodiments, the system comprises a microfluidic device comprising a plurality of single-entry, single-file cell growth trenches fluidically coupled to a growth channel; and a laser positioned to direct light at at least a portion of the cell growth trenches, wherein the laser is configured to produce light having an intensity capable of killing one or more cells, and a wavelength less than or equal to 1000 nm.

Another aspect is generally directed to a method. In one set of embodiments, the method comprises providing a microfluidic device comprising a cell flow layer that comprises a growth channel, a plurality of cell growth trenches containing cells fluidically coupled to the growth channel, and a control layer configured to control flow of the fluid in the cell flow layer; and

selectively killing cells contained within at least one of the cell growth trenches by exposing the cells to light at least sufficient to kill at least some of the cells. In some embodiments, the growth channel comprises an inlet portion, an outlet portion, an inlet valve portion associated with the inlet portion, and an outlet valve portion associated with the outlet portion.

According to another set of embodiments, the method comprises providing a microfluidic device comprising a cell flow layer comprising a growth channel, a plurality of cell growth trenches fluidically coupled to the growth channel, and a control layer configured to control flow of a fluid in the cell flow layer; selectively shielding, via a mask, cells contained within one or more of the cell growth trenches from light; and collecting at least some of the cells contained within one or more of the cell growth trenches. In some embodiments, the growth channel comprises an inlet portion, an outlet portion, an inlet valve portion associated with the inlet portion, and an outlet valve portion associated with the outlet portion.

According to another set of embodiments, the method comprises providing cells contained with plurality of single-entry, single-file cell growth trenches fluidically coupled to a growth channel; and selectively killing the cells contained within one or more of the cell growth trenches by exposing the cells to light at least sufficient to kill at least some of the cells.

According to yet another set of embodiments, the method comprises providing cells contained with plurality of single-entry, single-file cell growth trenches fluidically coupled to a growth channel; and selectively shielding, via a mask, cells contained within one or more of the cell growth trenches from laser light.

Other advantages and novel features of the present disclosure will become apparent from the following detailed description of various non-limiting embodiments of the disclosure when considered in conjunction with the accompanying figures.

### BRIEF DESCRIPTION OF THE DRAWINGS

Non-limiting embodiments of the present disclosure will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1 is a schematic representation of a side view of a system, according to some embodiments;

FIG. 2A is a schematic representation of a top view of one embodiment of a microfluidic device, according to some embodiments;

FIG. 2B is a schematic representation of a perspective view of the microfluidic device of FIG. 2A, according to some embodiments;

FIG. 2C is a schematic representation of a partially exploded view of the microfluidic device of FIG. 2B, according to some embodiments;

FIG. 3A is a schematic representation of a top view of a section of the microfluidic device of FIG. 2A in an unactuated state, according to some embodiments;

FIG. 3B is a schematic representation of a top view of a section of the microfluidic device of FIG. 2A in an actuated state, according to some embodiments;

FIG. 4A is a schematic representation of a side cross-sectional view of valve portions within a growth channel before actuation, according to some embodiments;

FIG. 4B is a schematic representation of a side cross-sectional view of valve portions within a growth channel during actuation, according to some embodiments;

FIG. 4C is a schematic representation of a side cross-sectional view of valve portions within a growth channel after actuation, according to some embodiments;

FIG. 5 is a flow chart illustrating a method for cell screening and retrieval, according to some embodiments;

FIG. 6A is a schematic representation illustrating the step of cell injection into the growth channel of FIG. 3A, according to some embodiments;

FIG. 6B is a schematic representation illustrating the step of flushing the growth channel of FIG. 3A, according to some embodiments;

FIG. 6C is a schematic representation illustrating the step of analyzing one or more cells of interest in the growth channel of FIG. 3A, according to some embodiments;

FIG. 6D is a schematic representation illustrating the step of selective killing of non-target cells within the growth channel of FIG. 3A, according to some embodiments;

FIG. 6E is a schematic representation illustrating the step of collecting and extracting cells of interest in the growth channel of FIG. 3A, according to some embodiments;

FIG. 6F is a schematic representation illustrating the step of cleaning the inlet and outlet portions of the growth channel of FIG. 3A, according to some embodiments;

FIGS. 7A-7C are schematic representations of a top view of a portion of a microfluidic device illustrating various steps of continuous cell extraction, according to some embodiments;

FIG. 8 is schematic representation of a microfluidic chip, according to some embodiments;

FIG. 9A is schematic representation illustrating the step of cell loading into the device of FIG. 8, according to some embodiments;

FIG. 9B is schematic representation illustrating the step of cell purging from the device of FIG. 8, according to some embodiments;

FIG. 9C is schematic representation illustrating the step of phenotyping cells trapped in the device of FIG. 8, according to some embodiments;

FIG. 9D is schematic representation illustrating the step of cleaning the device of FIG. 8, according to some embodiments;

FIG. 9E is schematic representation illustrating the step of selective killing of unwanted cells from the device of FIG. 8, according to some embodiments;

FIG. 9F is schematic representation illustrating the step of exporting cells of interest from the device of FIG. 8, according to some embodiments;

FIG. 10A is a schematic representation illustrating the laser ablation procedure; according to some embodiments;

FIG. 10B shows microscope images of pre-screening and post-screening of cells, according to some embodiments; and

FIGS. 11A-11C are schematic representations illustrating a method for continuous cell extraction using a continuous-culture and laser-ablation chip, according to some embodiments.

### DETAILED DESCRIPTION

The present disclosure is generally directed to systems and methods for retrieving cells from a continuous culture microfluidic device. In some aspects, a system that allows for selective extraction of one or more cells of interest from an arbitrary population of cells using a high-throughput negative cell selection technique is disclosed herein. For example, the system may comprise a microfluidic device comprising a plurality of cell growth trenches configured to contain cells and a patterned light source capable of selectively killing unwanted cells contained within the device. Coupled with time-lapse imaging, one or more cells of interest within the device may, in some aspects, be identified and extracted with a relatively high extraction efficiency, e.g., at least 99.9% of cells of interest may be extracted from the plurality of cells. In addition, some aspects of the disclosure are directed to methods for using such a system.

Cell screening play a fundamental role in biology and makes it possible to identify one or more cells of interest based on a desired phenotype and/or genotype. Various cell screening and retrieval methods have been developed over time. However, common challenges associated with conventional methods include low screening throughput, limited potential for scalability and automation, limited screening and extraction capabilities for complex cell populations, and phenotypic mischaracterizations caused by screening errors. Accordingly, certain aspects of the disclosure are directed to systems and methods that allow for high-throughput single cell screening and extraction with enhanced extraction efficiency.

The systems and methods described herein may have certain advantages compared to conventional systems and methods for cell screening and extraction. For example, certain embodiments of the system may include a microfluidic device configured to contain a plurality of cells and a dynamically patterned light source capable of selectively killing non-target cells. By destroying non-target cell lineages, one or more cells of interest may be retained and extracted. In some cases, the system may allow for the selective retrieval of target cells from highly complex cell populations (e.g., genetic libraries, clinical/ecological isolates) based on phenotypic and/or genotype profiling. Additionally, coupled with time-lapse imaging techniques, the system in some embodiments may allow for high throughput cell screening with a high cell extraction efficiency. The use of a light source (e.g., a laser) for negative cell selection may further impart the system with the potential for scale up and automation, in certain cases.

In some embodiments, a system configured for individual cell screening and retrieval is disclosed herein. In one set of embodiments, the system comprises a microfluidic device configured to contain a plurality of cells and a source of light (e.g., a laser) configured to selectively kill one or more of the plurality of cells contained within the microfluidic device. In some cases, such a system may be employed to screen live cells via a negative selection process. For example, the system may be employed to selectively kill cells (e.g., non-target cells) that exhibit one or more undesired characteristics (e.g., a genotype and/or a phenotype), such that cells (e.g., target cells) exhibiting one or more desired characteristics contained within a portion of the microfluidic device may be retained and retrieved.

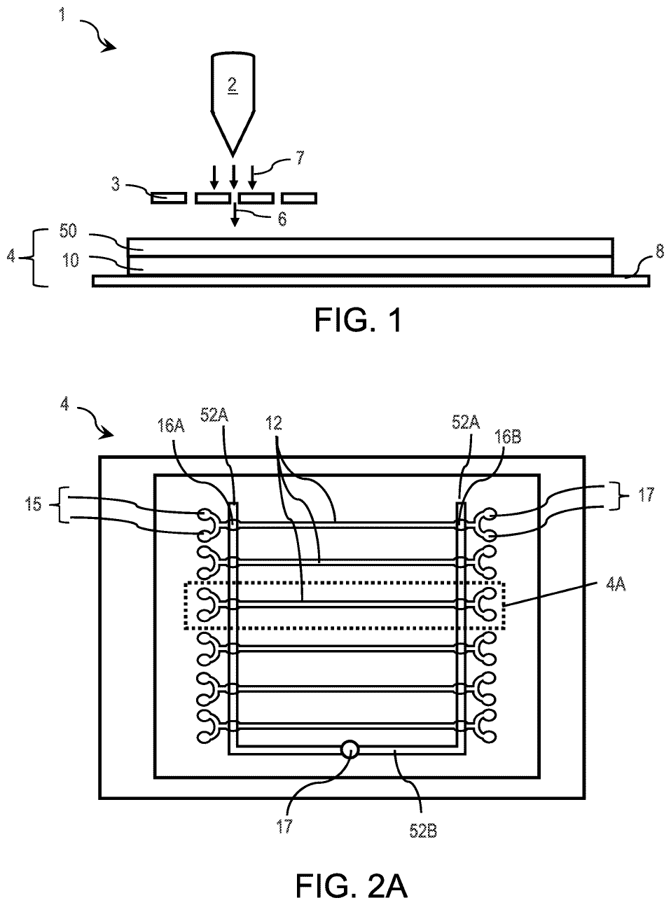

A non-limiting example of the system described above is shown in FIG. 1. As shown, a system 1 comprises a microfluidic device 4 and a light source 2 (e.g., a laser) positioned to selectively direct light at at least a portion of the microfluidic device. The light source, in some instances, may be configured to direct light (e.g., arrow 6 in FIG. 1) to a portion of the microfluidic device containing one or more cells. Accordingly, cells exhibiting one or more undesired characteristics may be selectively killed via that light, while cells exhibiting one or more desired properties may be retained within the portion of microfluidic device.

Additionally, in some embodiments, the system may further comprise a mask configured to selectively shield light from a light source directed at at least a portion of the microfluidic device. In some embodiments, the mask may be an electronically reconfigurable mask. In some embodiments, the mask may be positioned or configured to allow selective shielding of light directed at at least a portion of the microfluidic device containing one or more cells of interest. Advantageously, in some embodiments, the mask may prevent the one or more cells of interest from being exposed to the light source, such that the one or more cells may be retained during the cell-killing process described above.

FIG. 1 illustrates one embodiment of a system comprising a mask. As shown, system 1 comprises the microfluidic device 4, light source 2 (e.g., a laser) positioned to direct light at at least a portion of the microfluidic device, and mask 3 (e.g., an electronically reconfigurable mask) positioned to selectively shield a least a portion of the light directed at the portion of the microfluidic device 4. The mask, in some cases, may be configured to shield a portion 7 of the light originating from the light source 2, such that the portion of the microfluidic device underneath the mask is prevented from being exposed to light. As a result, cells residing within the portion of the microfluidic device may be shielded from light and thus retained.

The microfluidic device may have any of a variety of appropriate configurations described herein. The microfluidic device, in some cases, may have a configuration that allows for screening and extraction of individual cells.

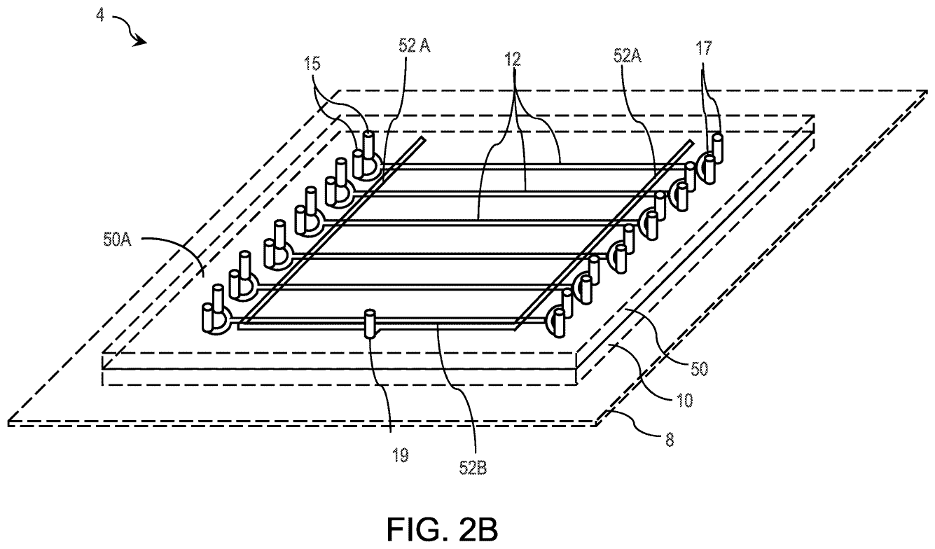

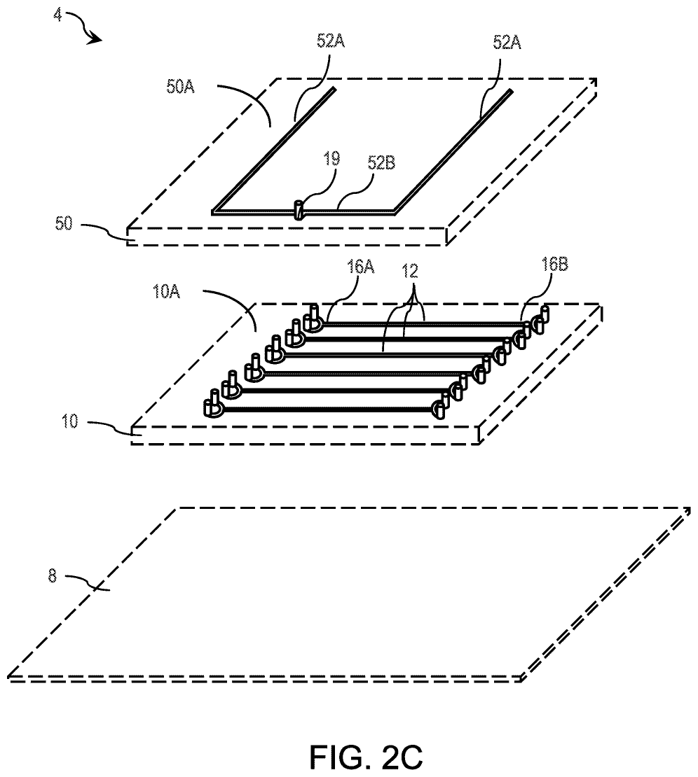

In some embodiments, the microfluidic device described herein (e.g., device 4 in FIGS. 1-2) may be employed for cell screening via multigenerational time-lapse microscopy. In certain embodiments, as described in more detail below, isogenic populations of cells may be confined and grown within the device, thereby allowing for the cells to be imaged or studied over many generations. As described in more detail below, one or more cells of interest can be extracted from the device via a negative cell selection and retrieval procedure in certain embodiments, and subsequently employed in a variety of downstream applications.

In some embodiments, the microfluidic device comprises two or more layers comprising microfluidic channels. For example, the microfluidic device may comprise a cell flow layer configured for flowing, receiving, and housing a plurality of cells. In some cases, the microfluidic device may further comprise a control layer positioned adjacent (e.g., coupled to) the cell flow layer. The control layer may be configured to control the flow of a fluid (e.g., cell media, a fluid comprising the plurality of cells, etc.) in the cell flow layer. In some embodiments, the microfluidic device further comprises a substrate (e.g., a coverslip) coupled to the cell flow layer, thereby forming a base layer of the microfluidic device.

A non-limiting example of the microfluidic device is shown in FIG. 1. As shown, the microfluidic device 4 may comprise a cell flow layer 10 and a control layer 50 positioned adjacent the cell flow layer 10. A substrate 8 may be coupled to the cell flow layer 10. forming the base layer of the microfluidic device 4.

While FIG. 1 shows one embodiment in which the microfluidic device comprises three layers, it should be noted that not all embodiments described herein are so limiting, and in other embodiments, the microfluidic device may comprise any additional layers disposed adjacent (e.g., directly adjacent) and/or between the one of more layers described above. In other embodiments, more or fewer layers may be present.

As noted above, the microfluidic device may include a substrate (e.g., coverslip), a cell flow layer coupled to the substrate, and a control layer coupled to the cell flow layer, in one embodiment. In some embodiments, the cell flow layer may include a variety of different channels through which cells (and other fluids such as growth media and cleaning fluids or solutions) can flow during use. The control layer may include various channels that can be filled with a fluid in order to actuate various different portions (e.g., valve portions) of the cell flow layer. As discussed in more detail below, the cell flow layer may comprise various portions (e.g., valve portions) that can be actuated by the control layer to selectively control the flow of cells and other fluids through the various channels of the cell flow layer. It should be understood, however, that flow layers, control layers, etc. in this example are but one method of confining or growing cells on a microfluidic device, e.g., for screening or other purposes, but that in other embodiments, other systems for confining or growing cells in a microfluidic device are also contemplated.

In some embodiments, the microfluidic device may comprise various channels having any of a variety of configurations and arrangements described herein. A non-limiting example of one embodiment is shown in FIGS. 2A-2C. As shown, FIGS. 2A-2C respectively illustrate a top down view, perspective view, and a partially exploded perspective view of the microfluidic device 4 of FIG. 1.

In one set of embodiments, the microfluidic device includes a cell flow layer comprising one or more growth channels configured to receive, flow, and/or house a plurality of cells. As shown in FIGS. 2A-2C, the microfluid device 4 may comprise a cell flow layer 10 comprising one or more growth channels 12. In some embodiments, the control flow layer comprises one or more control channels configured to control flow of a fluid in the cell flow layer. For example, as shown in FIGS. 2A-2C, the control flow layer 50 may comprise one or more control channels 52A configured to control flow of a fluid in the one of more growth channels 12 within the cell flow layer 10.

While FIGS. 2A-2C show a set of embodiment in which the microfluidic device comprises growth channels and control channels, it should be noted that not all embodiments described herein are so limiting, and in other embodiments, the microfluidic device may comprise various other types of channels in the cell flow layer and/or control layer. For example, as described in more detail below, the cell flow layer may further comprise one or more collection channels in additional to the growth channels.

In some embodiments, the channels (e.g., growth channels) in the cell flow layer are separated from the channels (e.g., control channels) in the control layer by an upper wall of the cell flow layer. See also Int. Pat. Apl. Pub. No. Wo 2020/257746. As shown in FIG. 2C. the various channels (e.g., growth channels 12) of the cell flow layer 10 may be defined on an underside of the cell flow layer 10. For instance, the cell flow layer 10 may include an upper wall 10A that forms the upper wall (e.g., the ceiling) of the various channels defined in the cell flow layer 10. When the cell flow layer 10 is bonded to the substrate 8, the substrate 8 forms a lower wall (e.g., a floor) of the various channels of the cell flow layer 10. Similarly, the various channels (e.g., control channels) of the control layer 50 may be defined on an underside of the control layer 50. An upper wall 50A of the control layer 50 may form an upper wall (e.g., a ceiling) of the various channels (e.g., control channels) of the control layer 50, and the upper wall 10A of the cell flow layer 10 may form a lower wall (e.g., a floor) of the various channels of the control layer 50.

In some embodiments, the channels of the cell flow layer and the control layer are fluidically coupled to the atmosphere via a plurality of vertical channels or ports. For example, as shown in FIGS. 2A-2C, the various channels (e.g., growth channel 12) of the cell flow layer 10 may be fluidically coupled to the atmosphere via a plurality of vertical channels extending upward through the cell flow layer 10 and the control layer 50 via one or more openings 15 and 17 defined in the upper wall 50A of the control layer 50. Similarly, the various channels (e.g., control channels 52A) of the control layer 50 may be fluidically coupled to the atmosphere via a plurality of vertical channels extending upward through the control layer 50 via one or more openings 19 defined in the upper wall 50A of the control layer 50. In some embodiments, the one or more openings may serve as inlets and/or outlet that allow for fluid flow into or out of the various channels in the control layer and cell flow layer.

In some embodiments, the cell flow layer comprises a growth channel comprising various portions, including an inlet portion, an outlet portion, and a main portion positioned between the inlet portion and the outlet portion. The growth channel may further comprise an inlet valve portion associated with the inlet portion and an outlet valve portion associated with the outlet portion. The various portions of the growth channel may be located at various positions along the length of the cell flow layer. For example, the inlet portion and the inlet valve portion may be located at a first end of the cell flow layer, while the outlet valve portion and the outlet portion may be located at a second end of the cell flow layer. The main portion may be located between the first end and the second end of the cell flow layer. Generally, each portion of the growth channel may function as a channel through which cells and fluids can flow.

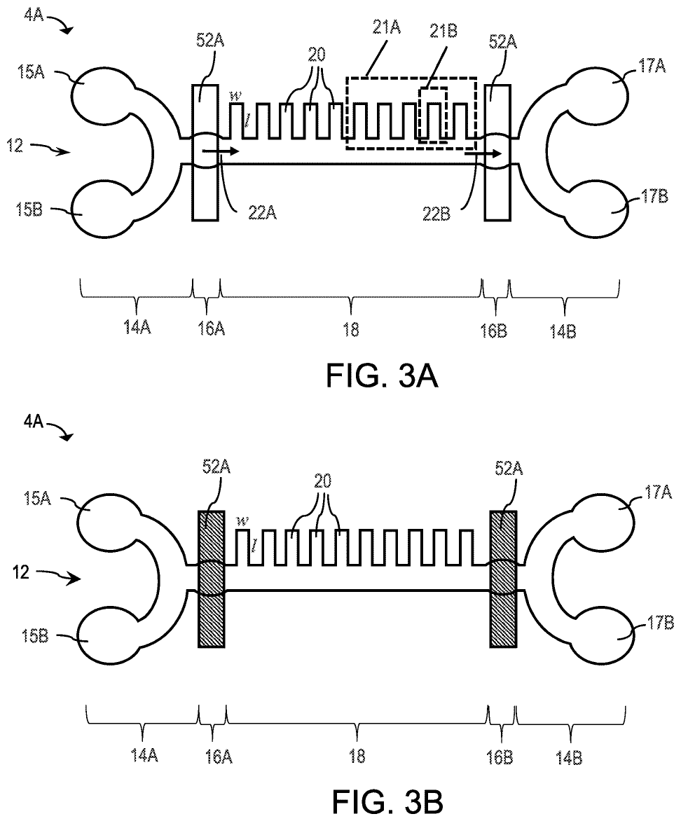

A non-limiting example of one embodiment of a growth channel is illustrated in FIG. 3A. Specifically, FIG. 3A shows a top down view of a section (e.g., section 4A) of the same microfluidic device illustrated in FIG. 2A. As shown, the growth channel 10 comprises various portions, including an inlet portion 14A at a first end of the cell flow layer, an outlet portion 14B at a second end of the cell flow layer, and a main portion 18 positioned between the inlet portion 14A and the outlet portion 14B. The growth channel 10 may further comprise an inlet valve portion 16A associated with the inlet portion 14A and an outlet valve portion 16B associated with the outlet portion 14B.

In some embodiments, the cell flow layer further comprises a plurality of cell growth trenches fluidically coupled to a main portion of the growth channel. In some embodiments, the plurality of cell growth trenches is configured to contain cells during use of the device. As shown in FIG. 3A, the cell flow layer may comprise a plurality of cell growth trenches 20 fluidically coupled to the main portion 18 of the growth channel 10. In some cases, the cell growth trenches may be positioned on a first side of the main portion of the growth channel, and may be configured to extend outward from the main portion of the growth channel in a direction that is perpendicular to the direction in which the main portion extends between a first end of and a second end of the cell flow layer. For example, as shown in FIG. 3A, the plurality of cells growth trenches 20 may be positioned on a first side of the main portion 18 of the growth channel and may extend outward from the main portion 18 in a direction perpendicular to a direction in which the main portion 18 extends. However, it should be understood that other arrangements of cell growth trenches are also possible in other embodiments. For example, the cell growth trenches may appear on both sides of growth channel, there may be more than one such growth channel, the cell growth trenches may independently be of the same or different sizes or lengths, the cell growth trenches may be symmetrically or asymmetrically arranged within the cell flow layer, or the like.

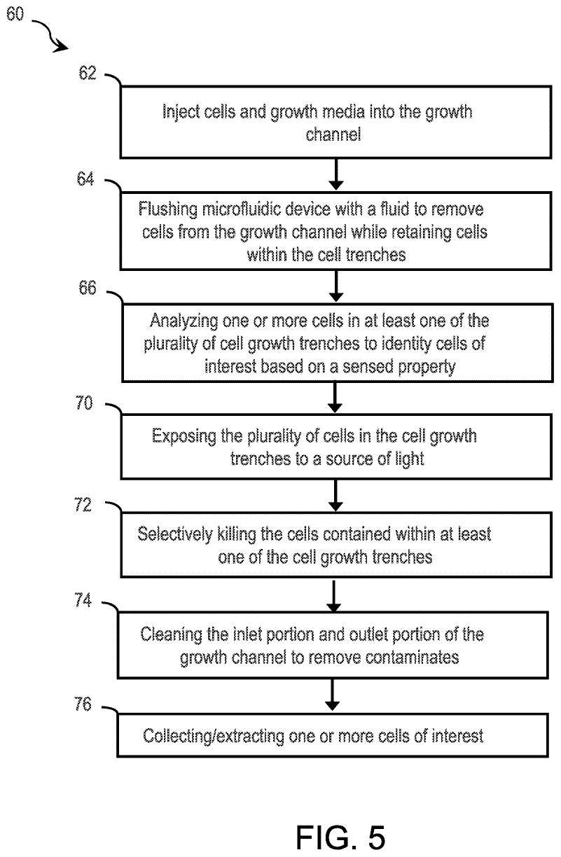

In some embodiments, the microfluidic device comprises a plurality of single-entry, single-file cell growth trenches fluidically coupled to the main portion of the growth channel. That is, some or all of the plurality of cell growth trenches may be sized so that individual cells are permitted to enter into the cell trenches one at a time in a linear, single-file fashion. In some cases, the cell growth trenches (e.g., growth trenches 20 in FIG. 3A) may have a width (e.g., width w in FIG. 3A) that is generally equal to or slightly larger than the width of individual cells entering into the growth trenches. For example, as shown in FIG. 5B, the single-entry, single-file cell growth trenches 20 may be sized such that individual cells are configured to fill the trenches in a linear, single-file fashion. In addition, in other embodiments, some or all of the plurality of cell growth trenches may be sized to allow more than one individual cell to enter at a time.

As noted above, the growth channel (e.g., 10 in FIG. 3A) may include, in certain embodiments, various valve portions, e.g., such as an inlet valve portion (e.g., 16A in FIG. 3A) and an outlet valve portion (e.g., 16B in FIG. 3A). In some embodiments, these valve portions may be actuated to aid in selectively controlling the flow of cells and fluid through the growth channel in the cell flow layer. For instance, the inlet valve portion of the growth channel may allow for control of fluid flow between the inlet portion of the growth channel and the main portion of the growth channel. Similarly, the outlet valve portion of the growth channel may allow for control of fluid flow between the main portion of the growth channel and the outlet portion of the growth channel. For example, as shown in FIG. 3A, the inlet valve portion 16A of the growth channel 10 may allow for control of a flow 22A between the inlet portion 14A of the growth channel 10 and the main portion 18 of the growth channel 10. Similarly, the outlet valve portion 16B of the growth channel 10 may allow for control of a flow 22B between the main portion 18 of the growth channel 10 and the outlet portion 14B of the growth channel 10. See also Int. Pat. Apl. Pub. No. Wo 2020/257746. However, it should also be noted that in other embodiments, there may be only a single valve portion present (e.g., an inlet valve portion or an outlet valve portion), and/or there may be no valve portions present.

In some embodiments, in the microfluidic device, the control layer may include one or more control channels configured to actuate the valve portions (e.g., inlet and/or outlet valve portions) within the growth channel in the cell flow layer. A variety of microfluidic valve configurations may be used in various embodiments.

A non-limiting example of one such embodiment is illustrated in FIG. 3A. As shown, the control layer (e.g., control layer 50 in FIG. 2) may include one or more control channels 52A coupled to (e.g., overlaps with) the inlet valve portion 16A and outlet valve portion 16B of the growth channels 10 in the cell flow layer (e.g., cell flow layer 10 in FIG. 2). The control channels 52A may be configured to aid in actuating the inlet valve portion 16A of the growth channel 10 and the outlet valve portion 16B of the growth channel 10.

The control channels may have any of a variety of appropriate configurations in the control layer. For example, in one set of embodiments, the control channel may have a U shape, as shown in FIGS. 2A-2C. As shown, the control layer may include one or more control channels 52A fluidically connected by a base channel 52B, thereby forming an overall U-shaped structure. In this embodiment, the control channels 52A may extend across (e.g., overlap with) the inlet valve portion 16A and outlet valve portion 16B of the growth channels 10. Specifically, the control channel 52A may overlap with portions of the upper wall 10A of the cell flow layer 10 that form the upper wall of the inlet valve portions 16A and outlet valve portion 16B.

While FIGS. 2A-2C illustrate an embodiment in which the control channels have an overall U shape structure, it should be noted that the control channels can have other shapes and/or configurations. In some embodiments, the control channels may extend across (e.g., overlaps with) all of the necessary valve portions of the cell flow layer. By having such a configuration, the control channel may serve as an on-off switch configured to close or open the valve portions of the cell flow layer. As described in more detail below, the control channels may be configured to actuate the valve portions to control fluid flow across the valve portions via pressurization. In addition, it should be understood that other valve configurations are also possible in other embodiments, for example, valve configurations that are able to control flow proportionally.

In some embodiments, one or more control channels in the control layer can be pressurized in order to actuate the valve portions (e.g., inlet and/or outlet valve portions) of the growth channels in the cell flow layer, for example, to serve as an on-off switch, or such that flow through the valve is proportional to the amount of pressure. As one non-limiting example, when the one or more control channels become pressurized, the inlet and outlet valve portions of the growth channels may be actuated to transition from an open state that allows for fluid flow through the valve portions to a closed state that prevents fluid from flowing through the valve portions. Conversely, when the one or more control channels become depressurized, the inlet and outlet valve portions of the growth channels may transition back from a closed state (i.e., actuated state) that prevents fluid from flowing through the valve portions to an open state (i.e., unactuated state) that allows for fluid flow through the valve portions.

FIGS. 3A-3B shows a non-limiting example of actuation of the inlet and outlet valve portions via the one or more control channels. As shown in FIG. 3A, when the one or more control channels 52A are not pressurized, the inlet and outlet valve portions 16A and 16B of the growth channels 12 remain in an open state that allows for fluids 22A and 22B to flow through the valve portions 16A and 16B. As shown in FIG. 3B, when the one or more control channels 52A become pressurized, the inlet and outlet valve portions 16A and 16B of the growth channels 12 become actuated and transition to a closed state that prevents fluid from flowing through the valve portions. Conversely, when the one or more channels become depressurized, the inlet and outlet valve portions 52A of the growth channels may revert from a closed state (as shown in FIG. 3B) to an open state (as shown in FIG. 3A).

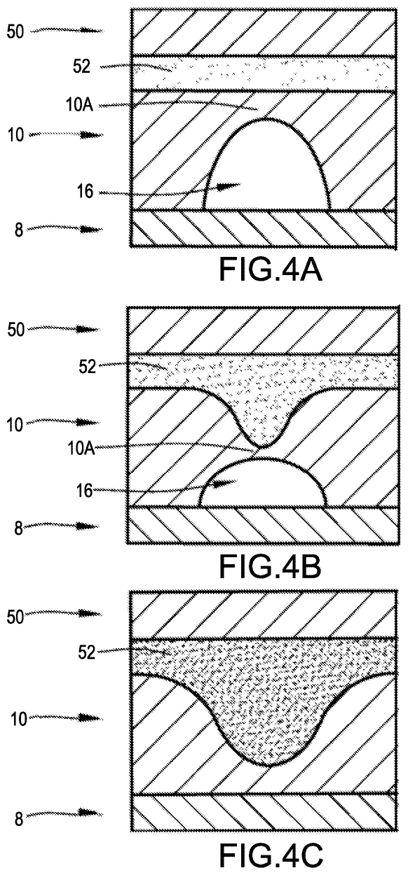

FIGS. 4A-4C show a cross-sectional view of another non-limiting example of a control channel in the control layer undergoing pressurization to actuate the valve portions of the cell flow layer. As shown, a control channel 52 (e.g., such as the control channel 52A in FIG. 3A) of the control layer 50 may be pressurized to actuate a valve portion 16 (e.g., such as the inlet and/or outlet valve portions 16A and 16B in FIGS. 3A-3B) of the cell flow layer 10 and thereby close off the underlying channels.

In FIG. 4A, the control channel 52 may be first filled with an incompressible or substantially incompressible fluid (e.g., distilled water or an aqueous solution, etc.). As shown, the control channel 52 is filled with a fluid, but has not yet been pressurized. Thus, the upper wall 10A of the valve portion 16 has not been compressed, and the valve portion 16 remain in an open state, thereby allowing fluid to flow through the valve portion 16.

In FIG. 4B, the fluid in the control channel 52 has begun to be pressurized (e.g., pressure has been applied to the control channel 52). Thus, the upper wall 10A and of the valve portions 16 has begun to compress and move toward the substrate 8. At this stage, the valve portion 16 has not been fully closed, and thus fluid can still flow through the valve portion 16.

In FIG. 4C, as more pressure is applied to the control channel 52, the upper wall 10A of the valve portion 16 may compress down and collapse toward the substrate 8. When this compression occurs, the valve portions 16 may transition to a closed state, such that no fluid may be able to flow through the valve portion 16. To return the valve portion 16 to its open state, the pressure is removed from the control channel 52. The material forming the upper wall 10A (which can be PDMS in some implementations) is generally elastic, such that the upper wall 16 can return to its uncompressed state (FIG. 4A) when the pressure is removed from the control channel 52.

In some embodiments, the valve portions described herein may have a cross-sectional shape that allows for efficient compression of the valve portions during actuation. As shown in FIG. 3A, in one set of embodiments, the valve portion16 generally has a dome-shaped cross-section. In some such cases, the dome-shaped cross-section aids may ensure that the upper wall at the valve portion can be easily compressed and does not resist the pressure from the control channel.

In some embodiments, the thickness of the upper wall of the cell flow layer at the valve portion may be relatively thin compared to the thickness of the upper wall of the cell flow layer at different locations. For example, as shown in FIGS. 4A-4C, the thickness of the upper wall 10A at the valve portions may be relatively thin, such that the upper wall 10A is flexible and can be compressed. At other locations (e.g., non-valve portions such as 14A, 14B, 18 as shown in FIGS. 3A-3B) along the growth channels of the cell flow layer, the upper wall may be relatively thick and the non-valve portions of the channels may have a different cross-sectional shape (e.g., a generally square or rectangular cross-section).

Referring back to FIGS. 3A and 3B, in order to actuate the valve portions 16A and 16B, the control channel 52A may be pressurized, such that the upper walls of the valve portions 16A and 16B are compressed to the substrate 8 and no fluid can flow through the valve portions 16A and 16B. Removal of pressure from the control channel 52A may return the valve portions 16A and 16B to their open states, such that fluid can again flow through the valve portions 16A and 16B.

In some embodiments, the first control channel 152A and 152B may always be filled with an incompressible fluid during use (whether the valve portions remain open or closed), and pressure may be applied to the filled control channels 52A to actuate the valve portions 16A and 16B. In some embodiments, all of the valve portions may be actuated simultaneously or near simultaneously by the control controls. In some embodiments, the incompressible fluid may be partially or wholly removed from the control channels 52A when the valve portions 16A and 16B are returned to their open states.

As noted above, the system described herein may comprise a light source (e.g., a laser) positioned to direct light at at least a portion of the microfluidic device. In some cases, the light source may be positioned to direct light at at least a portion of the cell growth trenches of the cell flow layer. For example, as shown in FIG. 3A, a light source (e.g., light source 2 in FIG. 1) may be positioned in the system to direct light at at least a portion 21A of the cell growth trenches 20 of the cell flow layer 10.

In some cases, a particularly beneficial type of light source having a certain intensity and/or wavelength of light may be employed in the microfluidic device. In one set of embodiments, the light source may have a visible wavelength spectrum. In one set of embodiments, the light source (e.g., UV laser) may have a wavelength in the ultraviolet regime. Non-limiting examples of light source that may be employed include, but are not limited to, a laser, a light-emitting diode, an arc lamp, etc.

In some embodiments, the light source (e.g., a laser) is configured to produce light having an intensity and/or wavelength capable of inhibiting or killing one or more cells. For example, in some embodiments, the light source may be configured to produce light having an intensity of greater than or equal to 0.1 W/cm², greater than or equal 0.5 W/cm², greater than or equal 1 W/cm², greater than or equal to 5 W/cm², greater than or equal 10 W/cm², greater than or equal to 50 W/cm², greater than or equal 100 W/cm², greater than or equal 500 W/cm², greater than or equal to 1,000 W/cm², greater than or equal 5,000 W/cm², greater than or equal 10,000 W/cm², or greater than or equal 50,000 W/cm². In some embodiments, the light source may be configured to produce light having an intensity of less than or equal to 100,000 W/cm², less than or equal 50,000 W/cm², less than or equal 10,000 W/cm², less than or equal 5,000 W/cm², less than or equal 1,000 W/cm², less than or equal to 500 W/cm², less than or equal to 100 W/cm², less than or equal to 50 W/cm², less than or equal to 10 W/cm², less than or equal to 5 W/cm², less than or equal to 1 W/cm², or less than or equal 0.5 W/cm². Any of the above reference ranges are possible (e.g., greater than or equal to 0.1 W/cm² and less than or equal to 100,000 W/cm², or greater than or equal to 100 W/cm² and less than or equal to 1,000 W/cm²). Other ranges are also possible.

In some embodiments, the light source (e.g., a laser) is configured to a produce light having any of a variety of appropriate wavelengths. In some embodiments, the light source (e.g., a laser) may have a wavelength of greater than or equal to 100 nm, greater than or equal to 125 nm, greater than or equal to 150 nm, greater than or equal to 180 nm, greater than or equal to 200 nm, greater than or equal to 225 nm, greater than or equal to 250 nm, greater than or equal to 275 nm, greater than or equal to 300 nm, greater than or equal to 325 nm, greater than or equal to 350 nm, greater than or equal to 375 nm, greater than or equal to 400 nm, greater than or equal to 420 nm, greater than or equal to 440 nm, greater than or equal to 450 nm, greater than or equal to 480 nm, greater than or equal to 500 nm, greater than or equal to 550 nm, greater than or equal to 600 nm, greater than or equal to 650 nm, greater than or equal to 700 nm, greater than or equal to 800 nm, or greater than or equal to 900 nm. In some embodiment, the light source (e.g., a laser) may have a wavelength of less than or equal to 1000 nm, less than or equal to 900 nm, less than or equal to 800 nm, less than or equal to 700 nm, less than or equal to 650 nm, less than or equal to 600 nm, less than or equal to 550 nm, less than or equal to 500 nm, less than or equal to 480 nm, less than or equal to 450 nm, less than or equal to 440 nm, less than or equal to 420 nm, less than or equal to 400 nm, less than or equal to 375 nm, less than or equal to 350 nm, less than or equal to 325 nm, less than or equal to 300 nm, less than or equal to 275 nm, less than or equal to 250 nm, less than or equal to 225 nm, less than or equal to 200 nm, or less than or equal to 180 nm, less than or equal to 150 nm, or less than or equal to 125 nm. The above-referenced values of wavelengths may have a deviation of +/−5 nm, of +/−10 nm, or +/−15 nm. Any of the above-referenced ranges may be possible (e.g., greater than or equal to 100 nm+/−5 nm and less than or equal to 1,000 nm+/−5 nm, greater than or equal to 200 nm+/−5 nm and less than or equal to 480 nm+/−5 nm, or greater than or equal to 250 nm+/−5 nm and less than or equal to 400 nm+/−5 nm). Other ranges are also possible.

The light source (e.g., a laser) may be capable of killing of one or more cells via any of a variety of appropriate routes. For example, in one set of embodiments, the light source may have an intensity and/or wavelength of light that is capable of killing the cells directly, e.g., such as damaging the DNA of the one or more cells. Additional or alternatively, as described in more detail below, the light source may have an intensity and/or wavelength of light that is configured to react with a chemical associated with the cells to produce a reaction product capable of destroying the cells. In some cases, one or more cells may be killed indirectly by a reaction product formed from a chemical or photochemical reaction.

As noted above, the system in some embodiments may comprises a mask (e.g., an electronically reconfigurable mask) positioned to selectively shield the light directed (by a light source) at at least a portion of the microfluidic device. The mask may be manually or automatically controlled. In some cases, the mask may be positioned to selectively shield light directed at at least a portion of the cell growth trenches in the cell flow layer. For example, light may be directed at the cells, that may be sufficient to inhibit or kill the cells. However, the light may not necessarily reach all of the cells in the device due to the presence of the mask, which may be able to selectively shield the incoming light. By positioning or configuring the mask appropriately, certain cells or portions of the device may be subjected to light (e.g., killing the cells in those portions) while other cells or portions of the device may be shielded from the light, e.g., such that those cells survive. As an illustrative example, as shown in FIG. 3A, the mask (e.g., mask 3 in FIG. 1) may be positioned in the system to shield at least a portion of the light directed at at least a portion 21A of the cell growth trenches 20 in the cell flow layer 10.

In some embodiments, the mask may be configured to selectively shield one or more cell growth trenches out of the plurality of cell growth trenches from light. For example, as shown in FIG. 3A, the mask (e.g., mask 3 in FIG. 1) may be configured to selectively shield at least one cell growth trench 21B out of the portion 21A of growth trenches that are exposed to the light. It should be noted that the mask may be employed to selectively shield any appropriate number of cell growth trenches and any particular cell growth trenches positioned along the main portion of the cell flow layer. This may be useful, for example, to control growth of cells within the growth trenches, for example, such that some cells are allowed to survive while other cells are not.

The system described herein may comprise any of a variety of appropriate types of masks. Non-limiting examples of masks include a spatial light modulator, a fixed aperture, a filter, etc. In some embodiments, the spatial light modulator comprises a micro-mechanical mirror-based spatial light modulator. In some cases, the spatial light modulator may include, for example, a digital micromirror device (DMD), a ferroelectric liquid crystal on silicon (LCOS) chip, a nematic liquid crystal (NLC) platform, a grating light valve (GLV), etc. In addition, in certain embodiments, more than one mask may be present, and the masks may independently be the same or different.

Certain aspects of the present disclosure are related to a method for selective cell screening and retrieval using the system and microfluidic device described herein (e.g., system 1 and device 4 in FIG. 1). In some embodiments, compared to conventional methods, the method described herein may be employed to extract one or more cells interest (i.e., target cells) with a relatively high extraction efficiency. The extraction efficiency may be determined by calculating a ratio of the number of target cells collected to the number non-target cells collected using a method described herein. For example, the method may have an extraction efficiency of at least 90% (e.g., at least 95%, at least 97%, at least 99%, at least 99.5%, at least 99.9%, at least 99.95%, at least 99.99%, at least 99.995%, at least 99.999%, or 100%).

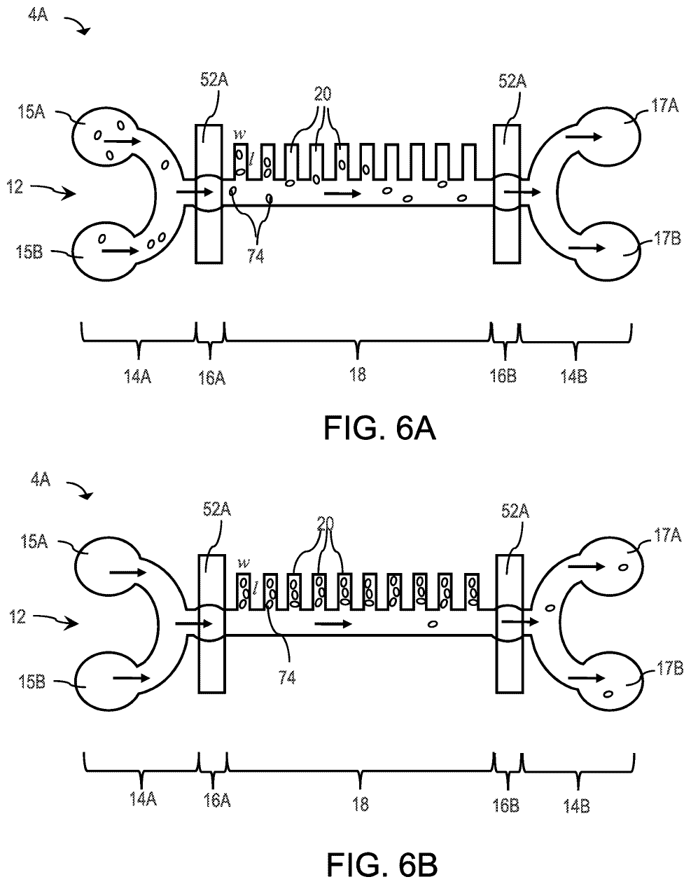

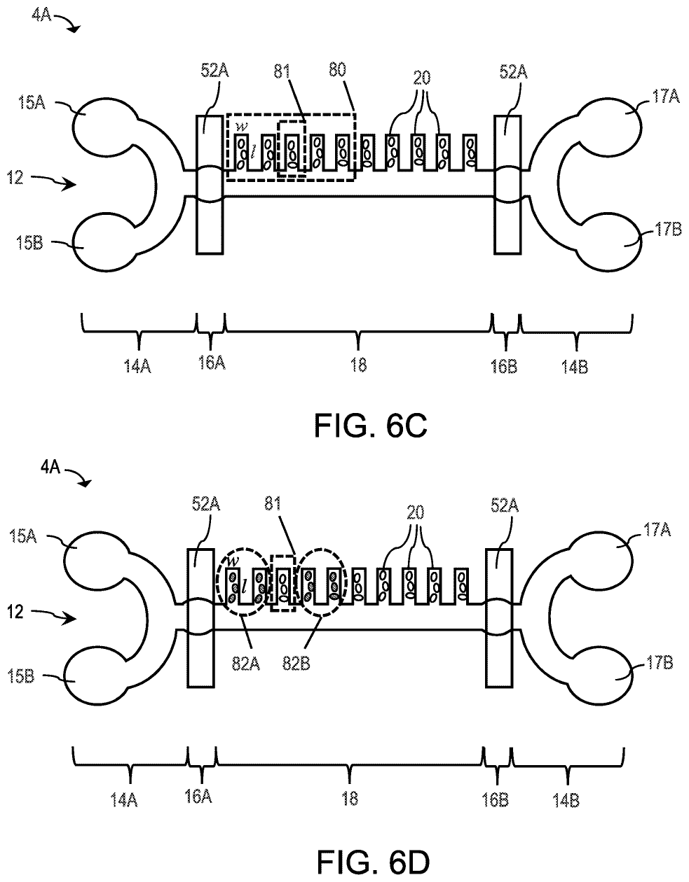

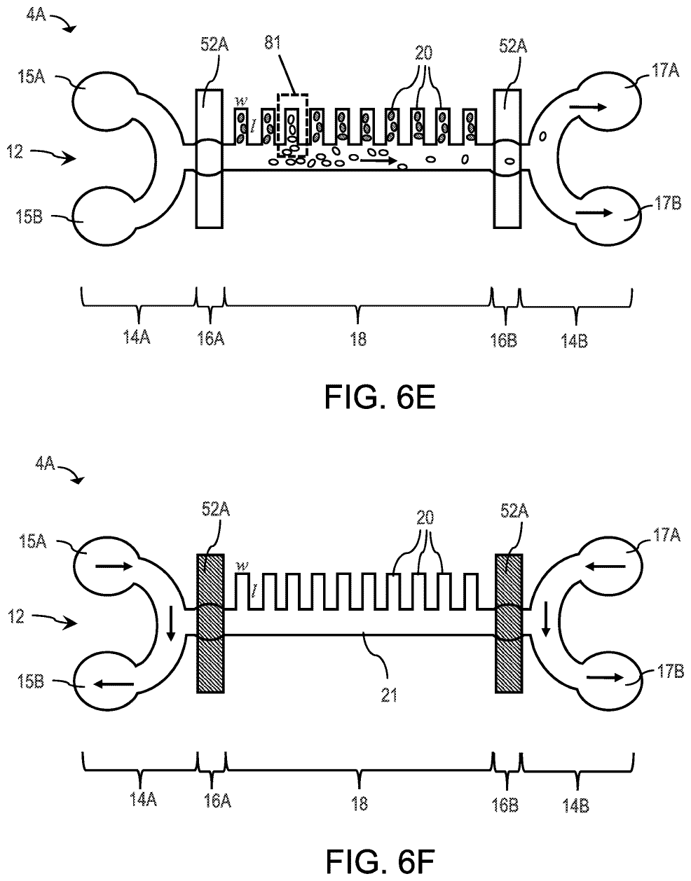

FIG. 5 illustrates a flowchart of a method 60 for one embodiment of selective cell screening and retrieval using the system and the device described herein. In addition, FIGS. 6A-6E illustrate various stages of the system and device at different steps of the method 60, in accordance with one embodiment. The system may have any suitable components described herein (e.g., microfluidic device, laser, mask, etc.), arranged in any suitable configuration. Certain non-limiting example configurations have been described previously with respect to FIGS. 1-4.

In some embodiments, the method comprises providing a microfluidic device, e.g., as discussed herein. As a non-limiting example, as noted above with respect to FIGS. 1-2C, the microfluidic device may comprise, in one embodiment, a substrate 8, a cell flow layer 10 coupled to the substrate 8, and a control layer 50 configured to control flow of the fluid in the cell flow layer 10. In certain embodiments, the cell flow layer may comprise a growth channel having an inlet portion, an outlet portion, an inlet valve portion associated with the inlet portion, a main portion, and an outlet valve portion associated with the outlet portion. The microfluidic device and associated components (e.g., cell flow layer, control layer, growth channels, control channels, cell growth trenches, etc.) may have any configurations and properties described previously. Non-limiting examples include those described with reference to FIGS. 1-4.

In some embodiments, the method comprises adding or injecting a fluid comprising a plurality of cells into the microfluidic device. See, e.g., step 62 in FIG. 5. In some embodiments, the cells and the growth media may be flowed into a main portion of the growth channel. For example, as shown in FIG. 6A, at step 702, cells 74 and growth media may be injected into the growth channel 12 through the inlet portion 14A. In some cases, the cells and growth media can be injected via one or more of the inlet openings. For example, as shown in FIG. 6A, cells 74 and growth media can be injected via one or more inlet openings 15A and 15B and flow into the main portion 18 of the growth channel 12.

Any of a variety of cells may be injected into the microfluidic device. Non-limiting examples of cells include bacteria cells, mammalian cells, bacteria, fungi, algae, protozoa, archaea, etc.

In some embodiments, during the injection step, the inlet and/or outlet valve portions of the growth channels are in an open state (e.g., an unactuated state). As noted above, in some embodiments, the control channels in the control layer are unpressurized. The control channels, when unpressurized, may allow fluid to flow through the valve portions of the growth channels. For example, as shown in FIG. 6A, when the control channel 52A is unpressurized, the inlet valve portions 16A and 16B are in an open state. The cells and growth media may thus able to flow through the inlet valve portion 16A into the main portion 18 of the growth channel 12.

In some embodiments, the cells injected into the main portion of growth channel may fill at least one of the plurality of cell growth trenches fluidically coupled to the growth channel. As shown in FIG. 6A, for example, the cells 74 injected into the main portion 18 may fill into at least one of the plurality of growth trenches 20. Any of a variety of methods may be employed to populate the cells from the main portion of the growth channel into the growth trenches. In some cases, for example, the cells may populate the cell growth trenches via diffusion. Additionally or alternatively, centrifugation may be employed to load the cell growth trenches.

In some embodiments, the method comprises providing cells contained with a plurality of single-entry, single-file cell growth trenches fluidically coupled to a growth channel. For example, after the injecting step, the plurality of single-entry and single-file cell growth trenches in the growth channel may be configured to contain the injected cells arranged in a linear, one-dimensional grouping (e.g., the cells are geometrically constrained to a single-file line). See, for example, growth trenches in FIG. 6B. In some cases, as cells that initially fill the cell growth trenches begin to divide, the cells grow into an isogenic lineage of cells within the plurality of cell growth trenches. These cells within a given trench may have the same genetic makeup, e.g., as they may have originated from the same original cell. In some embodiments, the cells may be arranged in a single one-dimensional line within a trench. For example, as shown in FIG. 6B, as the cells 74 in each of the cell growth trenches 20 begin to divide, each of the cell growth trenches 20 may eventually contains an isogenic lineage of cells. In some embodiments, if there is more than one cell type present within a cell growth trench (e.g., a plurality of cells having different genetic lineages), then the cells may be allowed to expand and divide. The cell closest to the closed end of the channel may divide to produce additional cells, which “push” the other cells out of the channel (e.g., the ones having different genetic lineages) such that eventually, only an isogenic lineage of cells is present within the cell growth trench, originating from the cell closest to the closed end of the channel.

In some embodiment, the method further comprises flushing the microfluidics device with a fluid, e.g., to remove the majority of cells from the growth channel while retaining one or more cells within at least one of the plurality of cell growth trenches. Referring again to FIG. 5 and FIG. 6B, as a non-limiting example, at step 64, after the cells 74 populates the one or more of the cell growth trenches 20, a fluid may be injected through the main portion 18 of the growth channel 12 to flush out the majority of cells from the growth channels 12 while retaining the one or more cells 74 in the cell growth trenches 20.

Any appropriate fluids may be employed to flush one or more of the growth channels within the device, in various embodiments. Non-limiting examples of suitable fluids include cell media, buffer solutions, etc.

In some embodiments, after populating one or more cell growth trenches with cells, the cells contained within at least one of the plurality of cell growth trenches may be analyzed to identify one or more cells of interest. One example is shown in step 66 in FIG. 5. For example, cells contained within a region of a growth trench may be analyzed using any of a variety of imaging techniques, in various embodiments. For example, the cells may be imaged using any number of different microscopy techniques including, but not limited to. light, fluorescence, phase contrast, bright field, light sheet, time-lapse microscopy, super resolution imaging, or any other suitable modality. In one set of embodiments, time-lapse microcopy may be advantageously employed, for example, to monitor and analyze various dynamic properties associated with the cells during the cell screening and extraction process.

In some embodiments, the method comprises identifying one or more cells of interest in one or more cell growth channels based on a sensed property. For example, one or more cells contained within a particular growth trench may be identified as cells of interest, e.g., based on a sensed property using imaging techniques described above. It should be noted the sensed property may include any property that can be directly or indirectly observed through various microscopy techniques, in accordance with various embodiments. For example, the sensed property may include, but are not limited to, a fluorescence property, a cell phenotype, a cell genotype, extracellular secretions, a marker or taggant associated with the cell (e.g., protein, antibody, etc.), etc. Specific examples of sensed properties include, but are not limited to, a cell type (e.g., bacterial strain identity, etc.), a cell morphology or physiology, gene expression, cell growth rate, cellular localization patterns, enzymatic activity, DNA replication and modification, chromosome segregation patterns, metabolic state, cell envelopes, spatial distributions of organelles, intracellular structures, cell-cell interactions, cellular secretions, or any combinations thereof.

In some embodiments, after identifying the one or more cells of interest from the plurality of cells in the cell growth trenches, the plurality of cells contained within the growth trenches may be exposed to a source of light (e.g., a laser light). For example, as shown in FIG. 5 and FIG. 6C, at step 70, the plurality of cells contained within the plurality of cell growth trenches 80 may be exposed to a source of light. In some embodiments, the source of light may be at least sufficient to kill at least some of the cells, e.g., such as non-target cells that are not identified as the one or more cells of interest. For example, the source of light may be at least sufficient to kill the non-target cells contained within a first cell growth trench, but not the cells of interest contained within a second cell growth trench.

In some embodiments, upon exposing the cells to a source of light, cells contained within at least one of the cell growth trenches may be selectively killed by exposure to the source of light (e.g., step 72 in FIG. 5). In one set of embodiments, the source of light (e.g., a laser light) may be directed (e.g., selectively directed) at cell growth trenches that do not contain the one or more cells of interest, such the cell growth trench that contains the one or more cells of interest is not exposed to the source of light. For example, as shown in FIG. 6D, a source of light (e.g., light source 2 as shown in FIG. 1) may be selectively directed at cell growth trenches 82A and 82B that do not contain cells of interest. Accordingly, cells contained with the growth trenches 82A and 82B that are exposed to the source of light may be killed and the cells of interest contained within cell growth trench 81 may be spared.

Any of a variety of appropriate methods maybe employed to selectively kill cells contained within the at least one cell growth trenches. In one set of embodiments, a patterned ablation light source (e.g., a patterned ablation laser light) may be employed during the cell killing process. For example, as shown in FIG. 6D, an ablation light source may have a patterned configuration that directs light simultaneously toward the non-adjacent growth trenches 82A and 82B, while not directing light at growth trench 81 positioned between the growth trenches 82A and 82B.

Additionally, in some embodiments, a mask (e.g., an electronically reconfigurable mask) may be employed to selectively shield the cells of interest contained in the one or more cell growth trenches from a source of light (e.g., laser light), for instance, is discussed herein. For example, as shown in FIG. 6D, a mask (e.g., mask 3 as shown in FIG. 1) may be employed to selectively shield the cell growth channel 81 containing cells of interest from the source of light. Thus, for example, the cells of interest contained within a first, masked cell growth channel may be spared while other cells contained within other, non-masked cell growth channels may be killed.

Cells may be killed due to a variety of mechanisms, in various embodiments. In some embodiments, for example, the step of selectively killing cells comprises selectively causing cells contained within at least one of the cell growth trenches to be killed via a chemical and/or photochemical reaction. In one set of embodiments, a chemical associated or contained with the cells may react with the source of light to produce a reaction product capable of killing the cells. For example, the cells may contain an exogenous chemical and/or an intracellular chemical capable of producing a cell-killing reaction product when exposed to the source of light. The reaction product may be configured to damage cellular components and comprise cell viability.

Non-limiting examples of chemicals capable of interacting with light to produce cell-killing reaction products include an intracellular porphyrin, a fluorescent dye, a chemical photosensitizer or protein photosensitizer, a chemical tag associated with the cells capable of interacting with light, etc. Non-limiting examples of photosensitizers include dyes (e.g., DNA-binding anthraquinone dye, DRAQ5, rose bengal, methylene blue, etc.), nanomaterials (e.g., quantum dots), etc. Non-limiting examples of reaction products capable of killing the cells include free radicals, reactive oxygen species (e.g., peroxides, superoxide, hydroxyl radical, singlet oxygen, alpha-oxygen, etc.), etc.

Generally, any number of cells of interest may be selectively retained (e.g., spared or extracted) in the plurality of cell growth trenches using the technique described herein. In some embodiments, a single cell of interest can be selectively retained from a single cell growth trench or from each of plurality of cell growth trenches. Alternatively, multiple cells of interest may be selectively retained from a single cell growth trench of from each of the plurality of cell growth trenches. In yet other embodiments, a single cell can be retained from each of a first set of one or more cell growth trenches, and multiple cells can be retained from each of a second set of one or more cell growth trenches.

In some embodiments, after selectively killing the cells contained within at least one of the cell growth trenches, at least some of the remaining live cells (e.g., cells of interest) contained within the one or more of the cell growth trenches may be collected and exported from the device (e.g., as shown in step 76 of FIG. 5). In some embodiments, prior to collection, some or all of the remaining live cells may continue to divide and fill at least a portion of the growth channel, e.g., as discussed herein. For example, as shown in FIG. 6E, after selectively killing the cells contained within at least one of the cell growth trenches 20. the remaining live cells contained within the cell growth trench 81 may continue to divide and grow into the main portion 18 growth channel 12. At least some of the remaining live cells contained within the cell growth trench 81 and the growth channel 12 may be subsequently collected. In some cases, a fluid may be then passed from the inlet portion 14A of the growth channel 12 through the main portion 18 of the growth channel 12 to flush the remaining live cells out of the outlet portion 14B of the growth channel 12.

In some embodiments, e.g., prior to collecting the remaining live cells, the method further comprises cleaning the inlet portion of the growth channel and outlet portion of the growth channel to remove contaminants (e.g., as shown in step 74 of FIG. 5). Such a cleaning step may advantageously prevent the remaining cells from being contaminated during the cell collecting step. While FIG. 5 shows a set of embodiment in which the cleaning step occurs after the step of selectively cell killing 72, it should be understood that not all embodiments described herein are so limiting, and in other embodiments, the cleaning step may occur any time between step 64 and step 76.

In some embodiments, the cleaning comprises closing an inlet valve portion of the growth channel and an outlet valve portion of the growth channel, e.g., such that the fluidic connection between the inlet portion of the growth channel and the plurality of cell growth trenches and the fluidic connection between the outlet portion of the growth channel and the plurality of cell growth trenches are disrupted. As noted above, the fluidic connections may be disrupted in certain embodiments when the one the inlet and/or outlet valve portions are actuated to transition to a closed state. In some cases, closing the inlet and/or outlet valve portion may comprise pressurizing one or more control channels associated with the control layer.

A non-limiting example of the cleaning step is illustrated in FIG. 6F. As shown, when the control channel 52A is pressurized, the inlet valve portion 16A and the outlet valve portion 16B may be actuated to transition to a closed state. Accordingly, the fluidic connection between the inlet portion 14A of the growth channel 12 and the plurality of cell growth trenches 20 and the fluidic connection between the outlet portion 14B of the growth channel 12 and the plurality of cell growth trenches 20 may be disrupted. The cells and the growth media remain in the main portion 18 and/or cell growth trenches 20 of the growth channel 12 may thus be prevented from flowing into the inlet portion 14A or the outlet portion 14B of the growth channel 12.

In some embodiments, after disrupting the fluidic connection between the inlet and/or outlet portion of the growth channel and the plurality of growth trenches, a cleaning fluid may be injected into the inlet portion and outlet portion of the growth channel to remove contaminates from the portions. For example, as shown in FIG. 6F, the inlet portion 14A may be cleaned by injecting a cleaning fluid into one or more of the first inlet opening 15A and the second inlet opening 15B. Since the control channel 52A is pressurized and the inlet valve portion 14A and/or the outlet valve portion 14B are closed, the cleaning fluid may flow through the inlet portion 14A or outlet portion 14B between the two inlet openings 15A and 15B or outlet openings 17A and 17B. The cleaning fluid may thus remove contaminants such as residual cells, growth media, and bacteria from the portions.

In some embodiments, cleaning the inlet portion and/or outlet portion may include employing a three-stage process, as described herein. For instance, a first cleaning liquid (e.g., bleach or an equivalent) may be first flowed through an inlet portion and/or an outlet portion to remove one or more contaminants, followed by a second cleaning liquid (e.g., alcohol (e.g., ethanol) and/or water, etc.) to remove the first cleaning fluid, and in some cases, subsequently followed by a growth media to remove excess second cleaning liquid and restore nutrient balance.

In some embodiments, after removing contaminates from the inlet and/or outlet portion of the growth channel, the inlet valve portion and the outlet valve portion of the cell flow layer may be opened to restore fluid connection between the inlet portion of the growth channel and the plurality of cell growth trenches and fluidic connection between the outlet portion of the growth channel the plurality of cell growth trenches. As noted above, opening the inlet and/or outlet valve portion may comprise depressurizing one or more control channels associated with the control layer.

Referring back to FIG. 6F, as a non-limiting example, once the inlet portion 14A and/or outlet portion 14B have been cleaned, the one or more control channels associated with the control layer 52A may be depressurized to open the inlet valve portion 16A and the outlet valve portion 16B. Accordingly, fluidic connection between the inlet portion 14A of the growth channel 12 and the plurality of cell growth trenches 20 and fluidic connection between the outlet portion 14B of the growth channel the plurality of cell growth trenches 20 may be restored. In some cases, additional growth media can be flowed into the main portion 18 of the growth channel 12 after the cleaning step.

In some embodiments, after the cleaning step, the cells in the cell growth trenches 20 may then be imaged, monitored, analyzed, exposed to a source of light, selectively killed, collected or extracted, as illustrated in FIG. 5. In some cases, as shown in FIG. 6A-6E, the inlet and/or outlet valve portions 14A and 14B of the growth channel 12 may be maintained in an open state during steps 62, 64, 66, 70, 72 and 76 of the method described in FIG. 5. It should be noted that in other embodiments, the disclosure is not so limited. For example, the inlet and/or outlet valve portions 14A and 14B of the growth channel 12 may be closed during at least one or more of the steps 64, 66, 70, and 72 of the method described in FIG. 5.

While FIGS. 4A-4C show an embodiment in which a microfluidic device having a particular configuration (e.g., microfluidic device 4 as shown in FIGS. 2-3) is employed in the system and method described herein, it should be noted that the disclosure is not so limited, and that in some embodiments, a microfluidic device having a different configuration may also be employed. Examples of such microfluidic devices are described, for instance, in International Patent Application No. PCT/US2020/038867, filed on Jun. 22, 2020, published as international Patent Publication No. WO2020257746 on Dec. 24, 2020, and entitled “Isolating Live Cells After High-Throughput, Long-Term, Time-Lapse Microscopy,” by Luro, et al., which is incorporated herein by reference in its entirety.

In one set of embodiments, a microfluidic device such as is described herein may allow for continuous cell extraction within the device. That is, in certain embodiments, a plurality of cells may be introduced into certain microfluidic devices to allow the cells to undergo at least two or more cycles of cell selection and retrieval prior to being collected from the device.

For example, in one set of embodiments, the microfluidic device may have a three-layered structure. One example, is shown in FIG. 1. In one set of embodiments, the microfluidic device may comprise a cell flow layer comprising two or more channels fluidically coupled to each other.

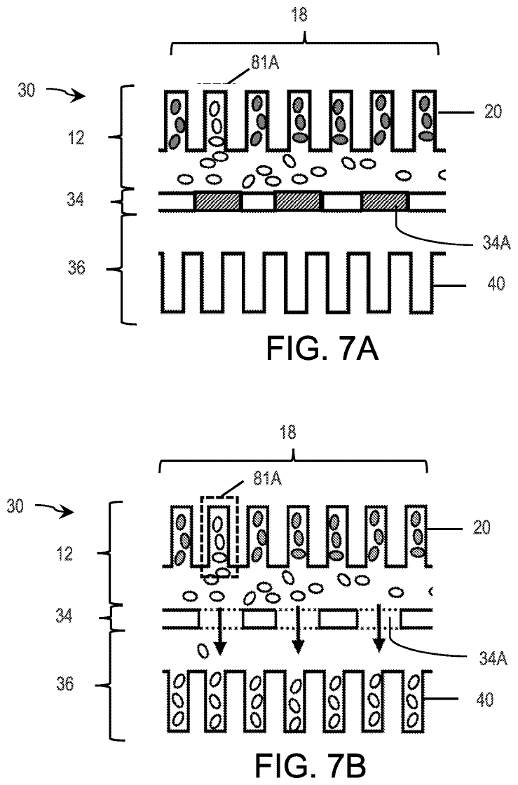

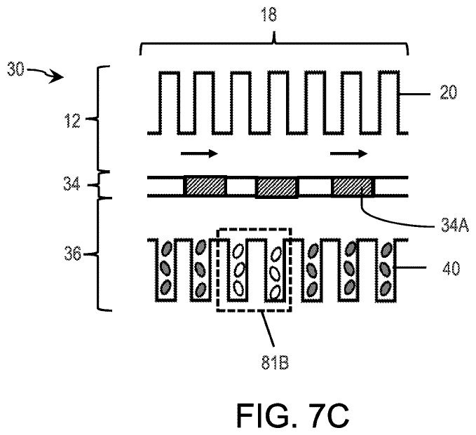

A non-limiting example of one such embodiment is illustrated in FIG. 7A. FIG. 7A is a schematic representation of a top view of a main portion of a microfluidic device 30 having two channel channels coupled to each other. As shown, the device may comprise a cell flow layer comprising two channels, e.g., such as a growth channel 12 and a collection channel 36, fluidically coupled to each other.

In some embodiments, one or more bridge channels may be positioned to couple the growth channel and the collection channel together. For example, as shown in FIG. 7A, one or more bridge channels 34 may be positioned to couple the growth channel 12 and the collection channel 36 together. The bridge channels may serve as pathways through which cells and fluids can travel from the growth channel to the collection channel.

In some embodiments, one or more of the one or more bridge channels may comprise a bridge valve portion configured to aid in selectively controlling flow between the growth channel and the collection channel. For example, as shown in FIGS. 7A-7C, the one or more bridge channels 34 may comprise one or more bridge valve portion 34A configured to aid in selectively controlling flow between the growth channel 12 and the collection channel 36. For example, when the bridge valve portion is in an open state, the growth channel may be in fluidic communication with the collection channels, e.g., as shown in FIG. 7B. When the bridge valve portion is in a closed state, the fluidic communication between the growth channel and the collection channels may be disrupted, e.g., as shown in FIGS. 7A and 7C.

In some embodiments, the bridge valve portion may be actuated to close or open by control channels located in a control layer adjacent the cell flow layer. The bridge valve portion may be actuated in the same manner as the inlet and/or outlet valve portions of the growth channel described herein (e.g., with respect to FIGS. 4A-4C). For example, the bridge valve portion may be actuated to close via pressurizing the control channels, and may be actuated to open via depressurizing the control channels.

The growth channel may have any of a variety of properties as described herein. For example, the growth channel may further comprise a plurality of single-entry, single-file growth trenches fluidically coupled to the main portion of the growth channel. In some embodiments, the collection channel may also comprise a plurality of cell growth trenches fluidically coupled to a main portion of the collection channel. For example, as shown in FIG. 7A, the collection channel 36 may comprise a plurality of cell growth trenches 40 fluidically coupled to a main portion of the collection channel 36.

The growth trenches associated with the collection channel may have any of a variety of properties, e.g., as described herein with respect to the growth trenches of the growth channel. The collection channel may also have a similar configuration and arrangement as the growth channel, e.g., such as an inlet portion, an inlet valve portion associated with the inlet portion, an outlet portion, an outlet valve portion associated with the outlet portion, a main portion between the inlet and outlet portion, various inlet and outlet openings, etc. Each of the above-referenced components of the collection channel may function in a substantially similar manner as the corresponding components associated with the growth channels.

The device shown in FIGS. 7A-7C can be used to illustrate a non-limiting example of continuous cell extraction. In some embodiments, a first cycle of cell screening and extraction may be carried out in the growth channel. As shown in FIG. 7A, the growth channel 12 has been subjected to the steps of cell injection, cell analysis, etc., as described in FIG. 5. For example, after identifying cells of interest based on a sensed parameter, the cells contained within the growth channel 12 has been subjected to a first cycle of selective cell killing (e.g., as illustrated by step 74 of the method 60 of FIG. 5). After the step of selectively cell killing, only the cells of interest contained within the growth trench 81A have been kept alive in growth trench 81A. In some embodiments, during the first cycle of cell screening and extraction, the control valve is pressurized to actuate the bridge valve portions 34A to a closed state. When in a closed state, the growth channel may be isolated from the collection channel such that there is no fluidic communication between the two channels.

In some embodiments, the remaining cells, after a period of cell growth and division, may be induced to flow from the growth channel across the bridge channels to the collection channel. As shown in FIG. 7B, for example, the remaining cells may flow from growth channel 12 across the bridge channels 34 to the collection channel 36. During this stage, the control valve may be depressurized to open the bridge valve portions 34A such that the growth channel is in fluidic communication with the collection channel.

In some embodiments, after flowing a majority of the cells has flowed from the growth channels across the bridge channels into the collection channel, fluidic communication between the growth channel and collection channel may be disrupted. For example, as shown in FIG. 7C, after flowing a majority of the cells has been flown across the bridge channels 34 into the collection channel 36, the bridge valve portions 34 may be actuated to close so as to prevent cells from leaving the collection channel 36 (as shown in FIG. 7C). The remaining cells may subsequently fill up the plurality of cell growth trenches 40 in the collection channel 36 in a single-entry, single-file manner.

In some embodiments, the cells contained with the plurality of cell growth trenches in the collection channel may be subjected to a second cycle of cell screening and extraction (e.g., as illustrated by steps 72-74 in FIG. 5). For example, as shown in FIG. 7C, after identifying cells of interest based on a sensed parameter, the cells contained with the plurality of cell growth trenches 40 in the collection channel 36 may be selectively killed, such that the cells of interest contained within the growth trenches 81B may be spared and extracted.

Depending on the application, any appropriate number of cell extraction cycles may take place in the device described herein. For example, as shown in FIG. 7C, in order for a third cycle of cell extraction to occur, the growth channel 12 may be cleaned to remove containments and any remaining cells. Additionally, the one or more bridge control valves 34A may be depressurized to transition to an open state, thereby re-establishing fluidic communication between the collection channel 36 and the growth channel 12. The remaining cells contained within the growth trenches 81B of the collection channel 36 may be flown across the bridge channels 34 into the cell growth trenches 20 of the growth channel 12. Accordingly, the cells contained with the cell growth trenches 20 may be subjected to another cycle of cell analysis and extraction, as illustrated by the steps in FIG. 5. It should be noted that during each cycle of cell screening and extraction, the cells of interest may be screened based on the same or different sensed property via any imaging techniques described herein.

The system and method described herein (e.g., system 1 as shown in FIG. 1) can be used in a variety of different applications. For example, a first application in which the system may be employed is to detect small but genetically stable differences in a wide range of properties of various cells. Most cell behaviors are statistically distributed such that a given genotype gives rise to a wide range of different phenotypes. For example, the expression of proteins in cells can vary substantially even between genetically identical cells growing in the same environment. In genetic screens, rare genetic variants with desirable properties are then often outnumbered by cells that only transiently display the desirable phenotype. The system may allow for tracking of each genetic variant for many generations of growth in multiple parallel cells and thereby provides a substantial statistical sample. The statistical sample allows genetically inherited traits to be separated from transient phenotypic variability, thus allowing the identification of rare variants of interest within large populations of cells that transiently mimic the interesting behavior.

In some embodiments, the system may allow for genetic variants of interest to be investigated without cloning the genetic variants of interest. Cloning is generally very time-consuming and resource-demanding, and may be difficult for complex genetic mutations across one or more chromosomes, plasmids, etc. The system may thus make cells of interest immediately available for further propagation, storage, downstream live-cell functional assays, and other applications.

In one set of embodiments, the system may be employed to identify epigenetic behaviors over long time-scales (e.g., many generations). Many cellular behaviors are epigenetic, e.g., the behaviors change on a time-scale of many generations. To identify such behaviors requires an observation window of multiple generations of cell growth, in some cases tens or even hundreds of generations. The system may allow for the retrieval of cells after observing these long-term epigenetic changes. The system may allow for application in a variety of processes, including genetic screens for chromatin remodeling, cell fate decisions, bistable circuits and multigenerational oscillators, or any other epigenetic behavior that can be observed through long-term imaging.

In one set of embodiments, the system may be employed to detect cell reactions to different environment. Many cellular behaviors depend on growth conditions. The system may allow for multigenerational imaging under many different environments, which allows the monitoring of changes associated with cells and cellular processes between environments. A large number of genetic variants can be monitored in parallel, and cells for variants of interest can then be extracted.

In one set of embodiments, the system may allow various assays to be performed. For example, by extracting cells physically, the system allows assays beyond DNA sequencing to be performed on the extracted cells, such as genome-wide terminal assays. By extracting the one of two daughter cells at each division, genome-wide time courses over cell lineages (for example tracking genome-wide properties) can be completed while simultaneously observing the properties of the daughter cells left in the system.

In some embodiments, the cell flow layer and/or the control layer comprise polymers (e.g., polydimethylsolixane (PDMS)) and may be cast together, or from separate molds. In some embodiments, the substrate is made from glass. The various channels of the cell flow layer and the control layer can be formed using any suitable fabrication technique(s). In some embodiments, the cell flow layer and the control layer are fabricated using multilayer soft lithography. In some embodiments, molds are initially formed from silicon wafers using UV lithography techniques. The cell flow and/or control polymer layers may then cast by flowing liquid polymer into the silicon molds, and then subsequently cured so that the polymer hardens. The two polymer layers can be bonded together (for example via curing or partial curing), and bonded to the substrate (for example via plasma bonding), and then further baked. Thus, the negative space of the channels of the cell flow layer and the control layer may be imprinted from the positive silicon wafer molds.

In some embodiments, the cell flow layer may have a length (e.g., such as a distance between the inlet and outlet of each channel in the cell flow layer) of between about 5 mm and about 100 mm, or about 30 mm; the control layer may have a span between various control channels (e.g., such as a distance between the two control channels 52A in FIG. 2A) of between about 4 mm and about 99 mm, or about 29 mm. The cell flow layer 10 and the control layer 50 may independently have a width of between about 20 micrometers and about 500 micrometers, or about 100 micrometers; the cell flow layer 10 may have a height of between about 5 micrometers and about 80 micrometers, or about 15 micrometers; and the control layer 50 may have a height of between about 10 micrometers and about 100 micrometers, or about 50 micrometers.

The cell growth trenches may have any of a variety of appropriate dimensions. In some embodiments, the length (e.g., l in FIG. 3A) of the cell growth trenches (e.g., the distance that the cell growth trenches 20 extend outward from the main portion 18 of growth channel 10) may be at least 1 micrometer, at least 5 micrometers, at least 10 micrometers, at least 25 micrometers, at least 50 micrometers, at least 75 micrometers, at least 100 micrometers, at least 200 micrometers, at least 300 micrometers, or at least 400 micrometers.

In some embodiments, the length of the cell growth trenches may be no more than 500 micrometers, no more than 400 micrometers, no more than 300 micrometers, no more than 200 micrometers, no more than 100 micrometers, no more than 75 micrometers, no more than 50 micrometers, no more than 25 micrometers, no more than 10 micrometers, or no more than 5 micrometers. Any of the above-referenced ranged are possible (e.g., between 1.0 micrometer and 500.0 micrometers). Other ranges are also possible.

As noted above, the width (e.g., w in FIG. 3A) of the cell growth trenches (e.g., 20 in FIG. 3A) may be comparable to the size of a single cell in certain embodiments. For example, the cell growth trench may have a width that is large enough fit a single cell. In some embodiments, the width of the cell growth trench may be at least 1 micrometer, at least 5 micrometers, at least 10 micrometers, at least 25 micrometers, at least 50 micrometers, or at least 75 micrometers. In some embodiments, the length of the cell growth trenches may be no more than 100 micrometers, no more than 75 micrometers, no more than 50 micrometers, no more than 25 micrometers, no more than 10 micrometers, or no more than 5 micrometers. Any of the above-referenced ranged are possible (e.g., between 1.0 micrometer and 100.0 micrometers). Other ranges are also possible.

In some embodiment, the height of the cell growth trenches may be at least 0.1 micrometers, at least 0.5 micrometers, at least 1 micrometer, at least 5 micrometers, at least 10 micrometers, at least 20 micrometers, or at least 40 micrometers. In some embodiments, the length of the cell growth trenches may be no more than 50 micrometers, no more than 40 micrometers, no more than 20 micrometers, no more than 10 micrometers, no more than 5 micrometers, no more than 1 micrometers, or no more than 0.5 micrometers. Any of the above-referenced ranged are possible (e.g., between 0.1 micrometer and 50.0 micrometers). Other ranges are also possible.

In some embodiment, the distance between adjacent pair of cell growth trenches (e.g., 20 in FIG. 3A) may be at least 0.1 micrometers, at least 0.5 micrometers, at least 1 micrometer, at least 2 micrometers, at least 4 micrometers, at least 6 micrometers, at least 8 micrometers, at least 10 micrometers, at least 20 micrometers, at least 30 micrometers, or at least 40 micrometers. In some embodiment, the distance between adjacent pair of cell growth trenches may be no more than 50 micrometers, no more than 40 micrometers, no more than 30 micrometers, no more than 20 micrometers, no more than 10 micrometers, no more 8 micrometers, no more 6 micrometers, no more 4 micrometers, no more 2 micrometers, no more 1 micrometers, or no more 0.5 micrometers. Any of the above-referenced ranged are possible (e.g., between 0.1 micrometer and 50.0 micrometers). Other ranges are also possible.

The cell growth trenches may have any of a variety of appropriate aspect ratios. For example, in one set of embodiments, the cell growth trenches may have a length (e.g., 1 in FIG. 3A) to width (e.g., w in FIG. 3A) aspect ratio of at least 2, at least 5, at least 10, at least 25, at least 50, or at least 75. In some embodiments, the cell growth trenches may have a length to width aspect ratio of no more than 100, no more than 75, no more than 50, no more than 25, no more than 10, or no more than 5. Any of the above-referenced ranged are possible (e.g., between 2 and 100). Other ranges are also possible.

International Patent Application No. PCT/US2020/038867, filed on Jun. 22, 2020, published as international Patent Publication No. WO2020257746 on Dec. 24, 2020, and entitled “Isolating Live Cells After High-Throughput, Long-Term, Time-Lapse Microscopy,” by Luro, et al., which is incorporated herein by reference in its entirety.

U.S. Provisional Patent Application Ser. No. 63/336,997, filed Apr. 29, 2022, entitled “Systems and Methods for Retrieving Cells from a Continuous Culture Microfluidic Device,” is incorporated herein by reference in its entirety.

The following examples are intended to illustrate certain embodiments of the present disclosure, but do not exemplify the full scope of the disclosure.

### EXAMPLE 1

This example describes a system and a method for scalable and precise screening of individual live cells following long-term time-lapse imaging, in accordance with certain embodiments.

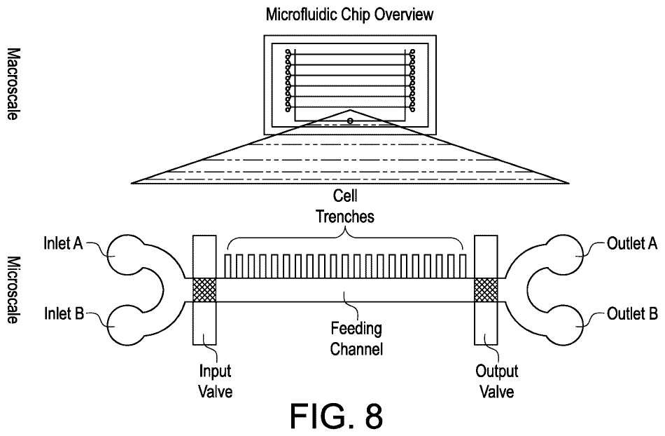

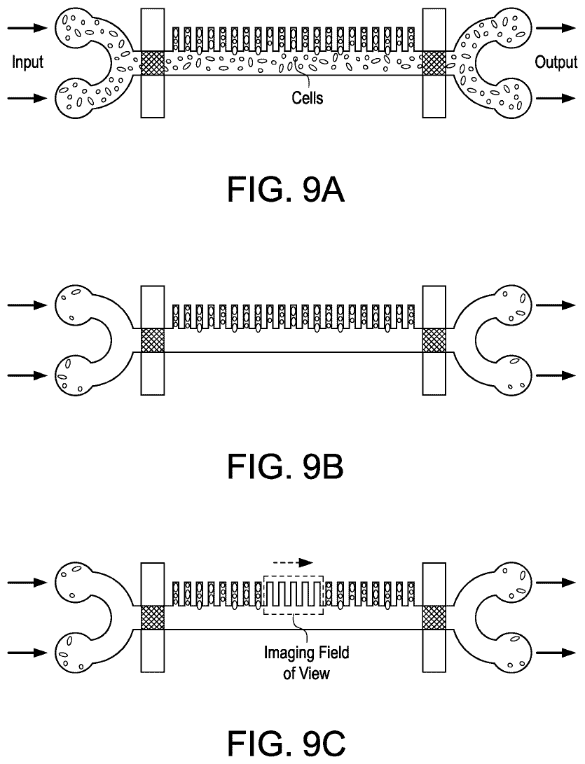

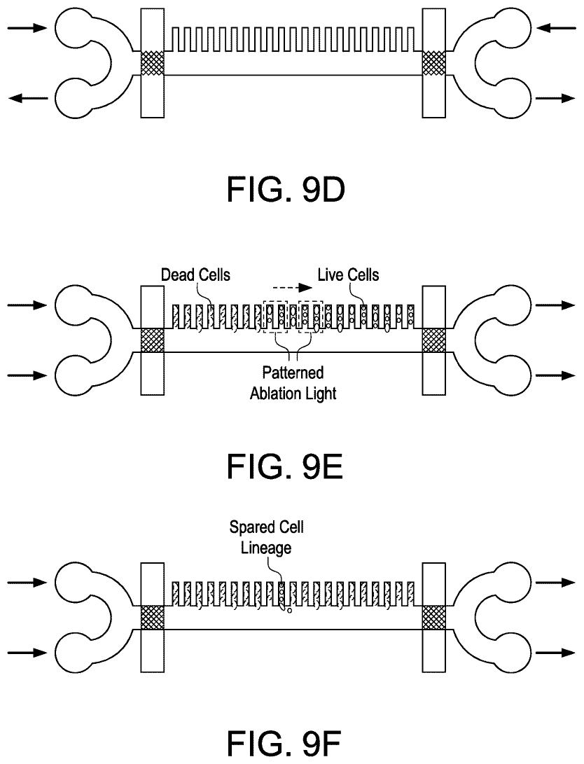

A microfluidic device as shown in FIG. 8 was employed for screening of individual cells. The method for screening of individual cells is shown in FIGS. 9A-9F, which respectively correspond to Step 1-6 of the method used in this example.

As shown in FIG. 9A, at Step 1, the microfluidic device was first loaded with cells. A cell suspension was flowed into the device through inlets A and B and then out of the device via outlets A and B. Cells populated the cell trenches by diffusion. The speed of the loading of the cell trenches may be accelerated by applying centrifugal forces (e.g., spinning entire microfluidic chip in microcentrifuge) and/or by concentrating the cell suspension prior to injection into the microfluidic chip.

As shown in FIG. 9B, at Step 2, the cells were purged from the feeding lane. Cell-free media was flowed into the microfluidic device via the same path as described in Step 1, to purge most cells from the feeding channel. The cells confined to cell trenches grew and divided as media was supplied via the feeding channel, continuously replenishing nutrients/drugs and washing away progeny cells. Different media may be applied to alter growth conditions.

As shown in FIG. 9C, at Step 3, the phenotype of cells in the growth trenches was analyzed using time-lapse microscopy. Time-lapse imaging was employed to characterize the phenotypes of cells trapped in cell trenches. Imaging may be used to characterize single-cell behaviors for an indefinite period of time, across either constant or changing media conditions.

As shown in FIG. 9D, at Step 4, the inlets and outlets of the device were cleaned. A stringent cleaning routine was employed to destroy all cells other than those housed in cell trenches, as described in more detail below. This process may be performed any time after Step 2 and before Step 6.

During Step 4, the inlet and outlet push-down valves were first pressurized, thereby pinching closed both ends of the feeding channel and disrupting fluid connection of the feeding channel and all growth trenches from the inlet and outlet regions. A sterilization solution (e.g., dilute bleach) was then flowed through the separated branched junctions, specifically into inlet/outlet A and out inlet/outlet B, respectively. This helped to remove all biofilms and residual cells leftover from device loading (Step 1) from the on-chip inlets and outlets as well as from all upstream connectors, tubing, and pumping/reservoir infrastructure, without harming cells restrained in cell trenches. The cleaning solution was then removed by flowing a washing solution (e.g., dilute ethanol) through the same junction paths as described. The wash solution was removed by flowing water through the same junction paths. Conditions in inlet/outlet junctions was then restored to those in the sealed feeding channel and cell trenches by flowing media through the same junction paths. Inlet and outlet push-down valves was then brought to atmospheric pressure and opened, thereby restoring fluid connection of the feeding channel and all cell trenches with the inlet and outlet regions.

As shown in FIG. 9E, at Step 5, unwanted cells was removed selectively by laser ablation. Unwanted cells (as determined by phenotypic characterization in Step 3) housed in cell trenches may be destroyed by patterned, high-intensity, laser light.

As shown in FIG. 9F, at Step 6, cells of interest was exported from the chip. The waste reservoir, receiving media exiting from outlets A and B, was changed to a clean receptacle. The non-targeted, surviving cells (e.g., those exhibiting phenotypes of interest) continued to grow and divide, exiting the cell trenches and entering the feeding channel. Cells were then exported from the microfluidic device and collected off-chip by flowing media through the feeding channel.

This non-limiting example illustrates a method that allows for the retrieval of single live cells from populations of arbitrary complexity (e.g., genetic libraries, clinical/ecological isolates) informed by high-throughput, long-term imaging of growing and dividing cells under tightly controlled conditions. Such screening is challenging to achieve using existing methods.

Furthermore, the method in this example is scalable and amenable to automation. The use of laser ablation may allow for greater screening throughput (e.g., more cells imaged per unit time) since denser microfluidic channel networks can be utilized. The ablation-mediated screening process also lends itself well to automation. The method described in this example can be paired in some embodiments with a digital micromirror device to dynamically pattern laser light and destroy unwanted cells, simply based on collected time-lapse imaging data. Automation of screening processes can be valuable to reduce cost, run-time, human input, and other scalability barriers.

The method described in this example may also allow for accurate cell retrieval. Using the method, it was demonstrated in single-cell collection from 10 individual cell lineages out of 5,000 total on-chip cell lineages without any errors or contamination. This accuracy showed a marked improvement compared with FACS-based methods to screen cells based on microscopy-resolved dynamics. Coupled with the duration of time-lapse imaging described above, which reduced screening errors due to phenotypic mischaracterization compared to even the conventional well-plate technologies, this method improved live-cell screening performance over start-of-the-art methods.

### EXAMPLE 2

This example describes a system and a method for retrieving individual live cells from a continuous-culture microfluidic device.

A platform for the isolation of individual live cells based on long-term time-lapse microscopy was previously developed. See International Patent Application No. PCT/US2020/038867, filed on Jun. 22, 2020, published as international Patent Publication No. WO2020257746 on Dec. 24, 2020, and entitled “Isolating Live Cells After High-Throughput, Long-Term, Time-Lapse Microscopy,” by Luro, et al. The method in this example makes use of optical traps to transport cells of interest from a contaminated cell-cultivation region of the device to a pre-cleaned aseptic compartment for export off chip. This example thus describes a method for cell retrieval. The approach in this example employed laser light to destroy all unwanted cell lineages, sparring only the lineage(s) of interest for outgrowth from the device.

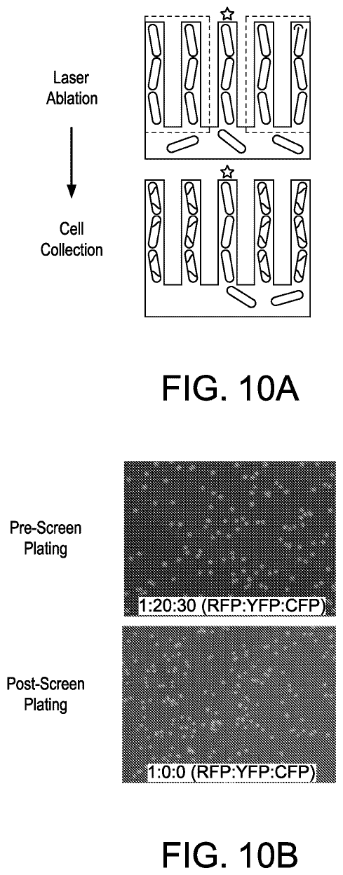

In order to reliably laser-kill bacterial cells in the microfluidic device, a chemical photosensitizer was used. Many excited fluorescent dyes, even those labeled for live-cell imaging, tend to react with molecular oxygen to produce free radicals that cause damage to cellular components and compromised viability. In this example, DRAQ5, a DNA-binding anthraquinone dye that intercalates between A-T bases of double-stranded DNA, was used because of its prominent photosensitization properties. Cells in the microfluidic device were first stained with DRAQ5 according to standard imaging protocols. Unwanted cells were then selectively illuminated with patterned 561 nm laser light (FIG. 10A). In FIG. 10A, all unwanted cells were ablated with patterned laser, while cells of interest continued to grow and are collected as they exit the device.

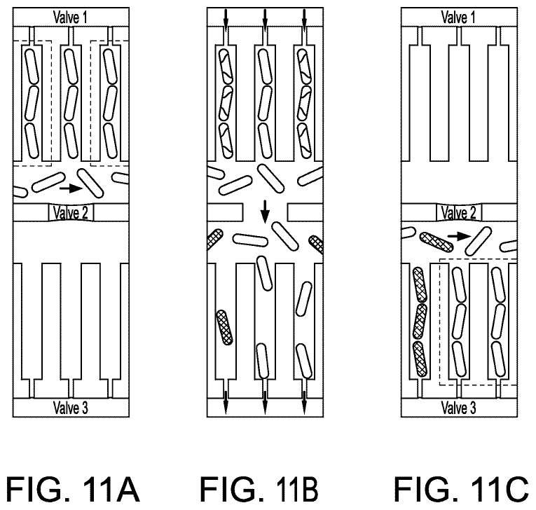

Microfluidic inlets and outlets were cleaned with valve-based procedures similar to those from optical trapping methods and outgrowth from the device was collected (FIG. 10B). FIG. 11B shows validation results for the laser-killing approach. As shown, a minority of RFP-expressing cells were isolated from the mother machine using laser ablation. The top panel shows colonies on an agar plate of cells collected from the microfluidic device, populated with a 1:50 minority of RFP-expressing cells, before screening with laser ablation. The bottom panel shows the same collection and plating after laser-killing-based screening for RFP-expressing cells. Both photos are composites of RFP, YFP, and CFP cannels. This example method thus demonstrated clean collection of targeted cells with laser killing.

It was demonstrated in this example that blue light (400-470 nm) could also be used to kill bacterial cells. This approach also triggers reactive oxygen species, but from photoexcitation of intracellular porphyrins instead of exogenous chemical photosensitizers. Indeed, using 405 nm laser light killed E. coli in the microfluidic device.

Laser killing, in some embodiments, may offer the use of pooled cell collections. In some embodiments, a laser killing approach can be used to achieve greater sampling throughput (i.e., characterization of larger input cell populations), e.g., by reducing the footprint of on-chip cleaning valve infrastructure. This may be due to the lack of a dedicated collection channel, as cells of interest may be harvested from the same fluidic passage used for cultivation and time-lapse imaging. This technique may also allow for simpler operation and may be more amenable to automation in certain embodiments, such as dynamically patterning light with digital micromirror devices. Laser killing could even allow for continuous artificial evolution on chip (FIG. 11). An example of continuous artificial evolution on chip is described below.

FIGS. 11A-11C are schematic representations illustrating a method employing a continuous-culture, laser-ablation, evolution chip. The microfluidic device may be operated by cycling through three general stages in this example.

In Stage 1 (FIG. 11A), a series of closed valves (represented by Valve 2) separate arrays of growth trenches perfused with growth media (top panel) from others in cleaning solution (bottom panel). Bacterial cells (ovals) may grow and divide within trenches supplied with orthogonally flowing media as in the conventional mother machine. Lineages may be characterized by time-lapse microscopy. Undesired variants may then be destroyed by patterned laser (dashed boxes).

In Stage 2 (FIG. 11B), the median valves may be opened. Spared variant(s) of interest may continue to proliferate on-chip and accumulate mutations (either naturally or via inducible mutagenesis machinery) and may be flushed into a second chamber (lower half of panel), randomly seeding clean trenches.

In Stage 3 (FIG. 11C), the median valves may be closed. The cycle may be resumed with cleaning the upper chamber, and time-lapse characterization and laser-selection within the lower chamber. Only a total of six growth trenches are shown for illustration purposes; the actual device may contain over one million.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or.” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of.” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the disclosure includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A system, comprising:
a microfluidic device comprising:
a cell flow layer comprising a growth channel having an inlet portion, an outlet portion, an inlet valve portion associated with the inlet portion, and an outlet valve portion associated with the outlet portion;
a plurality of cell growth trenches fluidically coupled to the growth channel; and
a control layer configured to control flow of a fluid in the cell flow layer;
a laser positioned to direct light at at least a portion of the cell growth trenches; and
an electronically reconfigurable mask positioned to selectively shield the light from the laser.

2. The system of claim 1, further comprising a substrate coupled to the cell flow layer.

3. The system of any one of claim 1 or 2, wherein the mask comprises a spatial light modulator.

4. The system of claim 3, wherein the spatial light modulator is a micro-mechanical mirror-based spatial light modulator.

5. The system of claim 3, wherein the spatial light modulator is a digital micromirror device (DMD).

6. The system of claim 3, wherein the spatial light modulator is a ferroelectric liquid crystal on silicon (LCOS) chip.

7. The system of claim 3, wherein the spatial light modulator is a nematic liquid crystal (NLC) platform.

8. The system of claim 3, wherein the spatial light modulator is a grating light valve (GLV).

9. The system of any one of claims 1-8, wherein the mask comprises a fixed aperture.

10. The system of any one of claims 1-9, wherein the mask comprises a filter.

11. The system of any one of claims 1-10, wherein the laser is configurable to produce light having a visible wavelength spectrum.

12. The system of any one of claims 1-11, wherein the laser is a UV laser.

13. The system of any one of claims 1-12, wherein the laser is configurable to produce light having a wavelength of greater than or equal to 100 nm and less than or equal to 1000 nm.

14. The system of any one of claims 1-13, wherein the laser is configurable to produce light having a wavelength of greater than or equal to 200 nm and less than or equal to 480 nm.

15. The system of any one of claims 1-14, wherein the laser is configurable to produce light having a wavelength of greater than or equal to 250 nm and less than or equal to 400 nm.

16. The system of any one of claims 1-15, wherein the microfluidic device further comprises one or more valves associated with the inlet valve portion and/or outlet valve portion of the growth channel, wherein the one or more valves are configured to control a flow of a fluid in the cell flow layer.

17. The system of any one of claims 1-16, wherein the control layer includes a control channel configured to actuate the inlet valve portion of the growth channel and the outlet valve portion of the growth channel.

18. The system of any one of claims 1-17, wherein the growth channel further comprises a main portion positioned between the inlet portion and the outlet portion.

19. The system of claim 18, wherein the inlet valve portion of the growth channel is configured to selectively control flow between the inlet portion of the growth channel and the main portion of the growth channel.

20. The system of any one of claim 18 or 19, wherein when the inlet valve portion of the growth channel is in a closed state, fluid is prevented from flowing between the inlet portion of the growth channel and the main portion of the growth channel.

21. The system of any one of claims 18-20, wherein when the inlet valve portion of the growth channel is in an open state, fluid is allowed to flow between the inlet portion of the growth channel and the main portion of the growth channel.

22. The system of any one of claims 18-21, wherein the outlet valve portion of the growth channel is configured to selectively control flow between the main portion of the growth channel and the outlet portion of the growth channel.

23. The system of any one of claims 18-22, wherein when the outlet valve portion of the growth channel is in a closed state, fluid is prevented from flowing between the main portion of the growth channel and the outlet portion of the growth channel.

24. The system of any one of claims 18-23, wherein when the outlet valve portion of the growth channel is in an open state, fluid is allowed to flow between the main portion of the growth channel and the outlet portion of the growth channel.

25. The system of any one of claims 18-24, wherein the plurality of cell growth trenches are fluidically coupled to the main portion of the growth channel.

26. The system of any one of claims 18-25, wherein at least some of the plurality of cell growth trenches are positioned adjacent to a first side of the main portion of the growth channel.

27. The system of any one of claims 1-26, wherein at least some of the plurality of cell growth trenches are configured to contain therein one or more cells.

28. The system of any one of claims 1-27, wherein the control layer is coupled to the cell flow layer.

29. The system of any one of claims 1-28, wherein the inlet portion of the growth channel includes a first inlet opening and a second inlet opening.

30. The system of any one of claims 1-29, further comprising a collection channel having an inlet portion, a main portion, an outlet portion, an inlet valve portion associated with the inlet portion, and an outlet valve portion associated with the outlet portion.

31. The system of any one of claims 17-30, wherein the control channel of the control layer overlaps with a portion of an upper wall of the cell flow layer that forms the inlet valve portion and/or outlet valve portion of the growth channel.

32. The system of claim 31, wherein a portion of an upper wall forming the inlet valve portion and/or outlet valve portion of the growth channel is configured to collapse toward the substrate in response to the control channel being pressurized with an incompressible fluid, thereby preventing fluid from flowing between the inlet and/or outlet portion of the growth channel and the main portion of the growth channel.

33. The system of any one of claims 30-32, wherein the main portion of the growth channel is positioned between the plurality of cell growth trenches and the main portion of the collection channel.

34. The system of any one of claims 30-33, wherein the inlet valve portion of the collection channel is configured to aid in selectively controlling flow between the inlet portion of the collection channel and the main portion of the collection channel, and wherein the outlet valve portion of the collection channel is configured to aid in selectively controlling flow between the main portion of the collection channel and the outlet portion of the collection channel.

35. The system of any one of claims 30-34, further comprising one or more bridge channels coupling the growth channel to the collection channel.

36. The system of claim 35, wherein at least some of the one or more bridge channels comprises a bridge valve portion configured to aid in selectively controlling flow between the growth channel and the collection channel.

37. A system, comprising:
a microfluidic device comprising:
a cell flow layer comprising a growth channel having an inlet portion, an outlet portion, an inlet valve portion associated with the inlet portion, and an outlet valve portion associated with the outlet portion;
a plurality of cell growth trenches fluidically coupled to the growth channel; and
a control layer configured to control flow of a fluid in the cell flow layer; and
a laser positioned to direct light at at least a portion of the cell growth trenches, wherein the laser is configured to produce light having an intensity capable of killing one or more cells, and a wavelength less than or equal to 1000 nm.

38. A method, comprising:
providing a microfluidic device comprising:
a cell flow layer comprising a growth channel having an inlet portion, an outlet portion, an inlet valve portion associated with the inlet portion, and an outlet valve portion associated with the outlet portion;
a plurality of cell growth trenches containing cells fluidically coupled to the growth channel; and
a control layer configured to control flow of the fluid in the cell flow layer; and
selectively killing cells contained within at least one of the cell growth trenches by exposing the cells to light at least sufficient to kill at least some of the cells.

39. The method of claim 38, further comprising injecting a fluid comprising a plurality of cells into the microfluidic device.

40. The method of claim 39, wherein injecting a fluid comprises injecting cells and growth media into an inlet portion of the growth channel such that the cells and the growth media flow into a main portion of the growth channel and fill at least one of the plurality of cell growth trenches.

41. The method of any one of claims 38-40, further comprising flushing the microfluidic device with a fluid to remove a majority of the cells from the growth channel while retaining one or more cells within at least one of the plurality of cell growth trenches.

42. The method of any one of claims 38-41, further comprising analyzing one or more cells in at least one of the plurality of cell growth trenches to identify one or more cells of interest.

43. The method of claim 42, wherein analyzing the one or more cells comprises performing microscopy on the one or more cells.

44. The method of any one of claims 38-43, after selectively killing the cells contained within at least one of the cell growth trenches, further comprising collecting remaining live cells from one or more of the cell growth trenches.

45. The method of claim 44, prior to the collecting step, further comprising cleaning the inlet portion of the growth channel and outlet/or portion of the growth channel.

46. The method of claim 45, wherein cleaning comprises closing an inlet valve portion of the growth channel and an outlet valve portion of the growth channel to disrupt fluidic connection between the inlet portion of the growth channel and the outlet portion of the growth channel from the plurality of cell growth trenches.

47. The method of claim 46, wherein when the inlet valve portion of the growth channel is closed, fluid is prevented from flowing between the inlet portion of the growth channel and a main portion of the growth channel.

48. The method of any one of claim 46 or 47, wherein when the outlet valve portion of the growth channel is closed, fluid is prevented from flowing between a main portion of the growth channel and the outlet portion of the growth channel.

49. The method of any one of claims 46-48, wherein closing an inlet and/or outlet valve portion of the growth channel comprises pressurizing one or more control channels associated with the control layer.

50. The method of any one of claims 45-49, wherein cleaning comprises injecting a fluid comprising one or more of a sterilization solution, a washing solution, and/or water into the inlet portion and outlet portion of the growth channel.

51. The method of any one of claims 45-49, wherein cleaning comprises opening an inlet valve portion of the growth channel and an outlet valve portion of the growth channel to restore fluidic connection between the inlet portion of the growth channel and the outlet portion of the growth channel and the plurality of cell growth trenches.

52. The method of claim 51, wherein opening the inlet valve portion and the outlet valve portion comprises depressurizing one or more control channels associated with the control layer.

53. The method of any one of claims 38-52, wherein the light arises from a laser.

54. The method of any one of claims 38-53, wherein selectively killing cells comprises reacting a chemical associated with the cells with the light to produce a reaction product capable of killing the cells.

55. The method of claim 54, wherein the reaction product comprises free radicals.

56. The method of any one of claim 54 or 55, wherein the reaction product comprises reactive oxygen species.

57. The method of any one of claims 54-56, wherein the chemical comprises an intracellular porphyrin.

58. The method of any one of claims 54-57, wherein the chemical comprises a chemical or protein photosensitizer.

59. The method of any one of claims 54-58, wherein the chemical comprises a chemical tag capable of interacting with light.

60. The method of claim 58, wherein the photosensitizer comprises one or more dyes selected from the group of a DNA-binding anthraquinone dye, DRAQ5, rose bengal, and methylene blue.

61. The method of any one of claims 58-60, wherein the photosensitizer comprises quantum dots.

62. The method of any one of claims 38-61, wherein the cells comprise bacterial cells.

63. The method of any one of claims 38-62, further comprising identifying one or more of the cells based on a sensed property.

64. The method of claim 63, wherein the sensed property comprises one or more of fluorescence, a cell phenotype, a cell genotype, extracellular secretions, and/or a marker or taggant associated with the cell.

65. The method of any one of claims 38-64, prior to selectively killing the cells, further comprising selectively shielding, using a spatial light modulator, cells contained within one or more of the remaining growth trenches from the light.

66. The method of any one of claims 38-65, wherein one or more cells in the at least one of the plurality of growth trenches are configured to grow into an isogenic lineage of cells.

67. The method of any one of claims 38-66, wherein the microfluidic device further comprises a collection channel fluidically coupled to the growth channel.

68. The method of claim 67, wherein the microfluidic device further comprises one or more bridge channels coupling the growth channel to the collection channel.

69. The method of claim 68, wherein at least some of the one or more bridge channels comprises a bridge valve portion configured to selectively control flow between the growth channel and the collection channel.

70. The method of claim 69, after selectively killing the cells, further comprising actuating, using the control layer, the plurality of bridge valve portions to allow fluid to flow through the plurality of bridge channels between a main portion of the growth channel and a main portion of the collection channel.

71. The method of any one of claim 69 or 70, further comprising closing, using the control layer, the plurality of bridge valve portions of the microfluidic device to prevent fluid from flowing through a plurality of bridge channels between a main portion of the growth channel and a main portion of the collection channel.

72. A method, comprising:
providing a microfluidic device comprising:
a cell flow layer comprising a growth channel having an inlet portion, an outlet portion, an inlet valve portion associated with the inlet portion, and an outlet valve portion associated with the outlet portion;
a plurality of cell growth trenches fluidically coupled to the growth channel; and
a control layer configured to control flow of a fluid in the cell flow layer;
selectively shielding, via a mask, cells contained within one or more of the cell growth trenches from light; and
collecting at least some of the cells contained within one or more of the cell growth trenches.

73. The method of claim 72, wherein the mask comprises a spatial light modulator.

74. The method of claim 73, wherein the spatial light modulator is a digital micromirror device (DMD).

75. The method of claim 73, wherein the spatial light modulator is a ferroelectric liquid crystal on silicon (LCOS) chip.

76. The method of claim 73, wherein the spatial light modulator is a nematic liquid crystal (NLC) platform.

77. The method of claim 73, wherein the spatial light modulator is a grating light valve (GLV).

78. The method of any one of claims 73-77, wherein the mask comprises a fixed aperture.

79. The method of any one of claims 73-78, wherein the mask comprises a filter.

80. The method of any one of claims 72-79, further comprising selectively causing cells contained within at least one of the cell growth trenches to be killed using a chemical and/or photochemical reaction.

81. A system, comprising:
a microfluidic device comprising a plurality of single-entry, single-file cell growth trenches fluidically coupled to a growth channel;
a laser positioned to direct light at at least a portion of the cell growth trenches; and
an electronically reconfigurable mask positioned to selectively shield the light from the laser.

82. A system, comprising:
a microfluidic device comprising a plurality of single-entry, single-file cell growth trenches fluidically coupled to a growth channel; and
a laser positioned to direct light at at least a portion of the cell growth trenches, wherein the laser is configured to produce light having an intensity capable of killing one or more cells, and a wavelength less than or equal to 1000 nm.

83. A method, comprising:
providing cells contained with plurality of single-entry, single-file cell growth trenches fluidically coupled to a growth channel; and
selectively killing the cells contained within one or more of the cell growth trenches by exposing the cells to light at least sufficient to kill at least some of the cells.

84. A method, comprising:
providing cells contained with plurality of single-entry, single-file cell growth trenches fluidically coupled to a growth channel; and
selectively shielding, via a mask, cells contained within one or more of the cell growth trenches from laser light.
